SEQUENCE LISTING (6 sequences)

SEQ ID NO: 1 - DNA, 64695 bp, Homo sapiens
aattcctcaacctccaaaactatgtatattttcagcttgatcaactatccttaaagtgtacctctttgaacaagataccggctgggtgcggtggctcatgcctgtaatcccagcactttgggaggccaaggcaggcagatcacctgaagtcaggagtttgagaccagccttgccaacatggtgaaaccctatctctactaaaaatacaaaaattagccgggcattgtggtgggcacctgtagtcccagctactcaggaggccgaggcaggagaatcgcttgaacccggaaggttaaggttgcagtgagccgagatggcgcagctgcactccagcctgggtgacagagtgaaactgtgtctcaaaaaaaaaaaagtcctgacacactggctcacgcctgtaatcctagcactttgggaggccaagacgagcagattgcctgaactcaggagtttgagaccagcctgggtaacatggtgaaaccccgtgtctactaaaacacaaaaaattagccaggggtcgcagtgtgcgcttgtagttccagctactcggaaggttgaagcagaattgcttgaacccaacaggcggaggtgcagtgagctgagatcatgccactgcactccagcctgggtgacagaggcaagactccgtctccaaaaaaaaaagataccaagtttcttagaggtataaaaaatacgttggtgtgccttggcgaggcgggaacacctttctagctccaaagtccagacccttacccactctattccacactgccacccttattcatgacacctgttgcacacaaactgagtcatggggaccagccatcattgctcgcagctgaggcctaaaaacgcgtaccctgcgtcccctccaccgccaccagccagcagtctgggaggcacccctagctcatgatgccgggtagaggaggggccgctgtttgcatgtgactctagcttcctcactgctaggctgtgcctaattattttatttttatttatttatctatctatttatttatttatttattgagacggagtcttgctctgtcgcccaggctcgagtgcagtagcgccatcttggctcactgcaagctccgcctcccgggttcacgccattctcctgcctcagcctcccaaggagctgggactacaggcgcccgccaccacgcccggctaattttttgtatttttaatagatacggggtttcaccgtgctagccaggatgatctcgatctactgacctcgtgatccgcccgcctcggcctcccaaagtgctgggattataggcgtgagccaccgcgcccggcggctgtggctaattattaggtaactagcgtcgttgggtctccctacctgaaggcctaaaggctggagataagagggctgaaggggagtagagccaggactggtgggcctgggagccaccaatagtcccagaagatcctacggggactttgccttctgctcctgatctctggaatcgattcatcagcctgagcttcgactccagcctctgcatccgcttctcccaaggtgattcctcaatgtctgctctgggcacctcatggatgcatcgcaagggctttagggagtcctctgctcacagggattgacctacccgcttgccaggtctcaagctcttgagagaaagcctgatgctctttttgtcttcacctccatcctaggcactgggtaggaacacagccaagaacgattgcaggatgggtccttccaggacactgacgtctcagcttgcgcactgtgagtccctggacgagttactccacctctctgaacctcctcctcacttgcataatgggaaaaataatggacataggaagatgaaacaagaccttggagaccacatttaccatcttattttccttgttcctttttcttttctttttttgtttttgtttgtttgttttttggagacagggtctcactctgtcacccaggctggcgtgcaatggcgcgatctcagctcactgcagcctctgcctcctgtgttcaagggatcctcccacctcagcctcccgggtagctggcaccacagacgcacaccaccatgcctggctaaatttttttgtatttttagtagaagtggggttttaccatgttggccaggctgttctcgaactcctggcctcaagtgatccgtccacctcggcctcccaaagtgctgggattacaggcgtgaaccactgagcccggccccttgttccctgttctgtttctttttcactgttatcactgatcaccatcgatcatattatctacatatctattagattggcgcaaaagtaattgctgtttttgccattacctccaatggcattactttcagtggcaaaaactgcaattacttttgcgccaacctaatatattgagacagggtctctctctgtcacccaggctgctggagggcagtgacacaatcacagctcactgccatgtccaactcctggattcaagtgatcctcccgcctcagtctcctgattacctgggactacaggcatgtatcaccacacccagccggtttttttgtttgtttgtttttgaaacagttttgcttttgccccccaggctggagtgcaatggcatgatcttggttcactgcaacctctgcctccggggttcaagcgattctcctgtctcagcctcccaagtagctgggattacaggtgcctaccaccacacctggctaatttttggttttgtgggtgtgtgtgtgtgtgtgtgtgtgtgtgtgtgtgtgtgttggagtctggctctgtcacccaggctgctggagtgcaatggcgcaatcttcagctcactgcaacctctgcctcctgggtttaagcaattctcctgcctcagactcctgagtagctgagattacaggtgtccaccaccacgcccggctaatttttgtatttttagtagagacagggtttcaccgtgttggccaggctggtctccatctcctgacctcaggtgatccacctgcctcagcctcccaaagtgctgggactacaggcgtgagccaccacgcccggctcaagtttctctttttctttttttttttttacattttgtagagacagggtctcactatgttgcccaggctggtcttgaactcctgatctcaagcaatcctccctctctggctcccgaagtgcggggataataggcatgaaccactgtgcacagcccatattttatattctacttaactgagtttctttagtttttattcctcccactaggatgtctgttttgtttacctccatatctccagcccctagaacagaacttggttcatggtgggttcagtaactatttgaattaaatgaatagatggacgtaaaatggttagtactgcggtgcccagtaatggtaggtatcaaacttagtctctgtgctccaacagaaagcagtgtaatttaagatgggcacagtgggctgggcgcggtggctcacacctgtaatcccagcactttgggaggccgaggcaggtggatcacctggggtcaggagttcgagactggcctggccaacatggtaaaaccttgtctctactaaaaatacacaaaattagctgggtgtggtggtggtcacctgtaatcccagctacttgggaggctgaggcaggagaattgcttcaacctgggaggtggaggttgcagtgagccaagatcacgccactgcactccagcctgggcgacagagcgagactccgtctcaaaaaaaaaaaaagatgggcacagtggctcccgcctgtaatcccagcactttgggaggccgaggtgggcagatcacccgaggtcaggagttcgagaccagcctggccaacatggtgaaaaaccctgtctctactaaaaatacaaaaattagccgggtatggtggcacacgcctatagtcccagctacttgggaggctgaagcaggagaatcacttgaacctgggaggcagagattgcagtaagccaaaatctggccactgcactccagccagggtgacagagccagactccatctcaaaaaaaaaagaacgaaagaaagagagcagtgtaattggagcagggaaaatgcagcatcgtaataggaggtacaggttaggagctctggctgagaaatcccgtctgtaccatgtaccagctgtgtgacctggaacagaagaatgaattcaccttcctgaggctcagtctcctcatctgtcaaatgcaggtgatgtaggacctcctgggaaggatgtgagcatgcaggagatggtaaatgaaaagcacttagcagagtgtctggtgcataataagtgctcagaaaacactggcctacgctgttcattatagtaatactcagatgagggggacctctgagtcaattaaacagttgctaagcacttaaaatatgtcacgggccagcgagtccagtaaacatttattgacttaaatctgtgaactccttaagggaagggactgtgtctacattgtcatgatcccggccccagaagatgctaattaatgtgaaaacatgctggaaaagttacatactgaactcaaatatgagttagaatacagaatagctattagagtaataacacccattgcctaacatgtgccaggcttgctgttgggtgttggagaaagggcagggagcttaaacaaacatgggaacttacagcttagcaggggagacagactttgttctgagaattgcacagatagagaggaaactctgaagttgataaatatcacaaagaagacatatgtggtgctgtgagttccataatatggagggagggagggttgacctggtctcggactggggagggagtgatgcctagagctgagctctgaagaaaagttctttgctaggctgagtgttggggaagtgctctcagcagatgaaatggcagatagaataggccttctgactgaggcacacatgtgctaaagggcgcaaaagaaggcccatatgacacaagcatagtgagctgagggtgcgattgggggtgaaataaactgcaaaaattagtagacactttgggaactagctgggcaatgtctgataaagctgaaggtgtgtgtaacctatgatgcagccgctccacagctgaggacattgttgacagaaatgcaggagtgcactgggaagcatgaacaagaatgatggccctagcagcattgtatatcatcacaaaaaactggaaataggctgggcgcggtgactcatgcctgtaatcccagcactttgggaggccaaggcaggcagatcacctgaggtcaggagttcgagaccagcctggccaacacggtgagaccccatctctactaaaaatacaaaaattagccgggtgtggtggcacatgcctataatcccagctactcgggaggctgatcaggagaactgcttgaacccgggaggcggaggttgcaatgagccaggatcgtaccactgcactccagcctggggcaacagagcaagactttgtctcaaaaaaaaaaaaaaaaaaaaaaaaaagacaggttcaggcctggtgtggtagctcacatttgtaatgccagcactttaggaggccaagacaggagaatcgctttagtccaggagtttgagaccagcctgggcaacttagtgagactctgtctctgcaaaaaaaaatgtaaaagtatgggcgcggtggctcatgcctgtaatcccagcattttgggaggccgaggcaggtggatcacctgaggtcaggagttcgagaccagcctggccaacatggcaaaaccccatctctactaaaaatacaaaaatttgctgggcatggtggtgtgtgcctgtaatcccagctactcaggaggctaaggcaggagaatcacttgaacctgggaggcggaggttgcagtgagccaagattgcaccattgcactccagcctgggcaacaagagcaaaactccatctcaaaaagaaaaatgaaaaaaattagctgggtgtggtggtgcactcctgtggtaccagtttctctggaagctaaggtgggaggatcacttgagcccacaaaatcaaggctgcagtgagccatgatcgcaacactgcactccagcctgggcaacagagtgagaccctgtctccaaaaaaaaaaaaaaaaaaaaattctgggcacggtggttcacgcctgtattcccagcactttgggaggcccaggcaggtggatcacttgaggtcaggagttcaagaccagcctggacaacaggctgtattttgtattttgtctattaaaaatagaaaaattagctcggcatggtggcgtgtgcctgtaattccagctacttgggaggctaaggcagagttacttaaatctgggaggtggaagttgcagtgagctgagatcgcgccactgcactccagcctgggcgacagggagactccatctcaaaaaaataaaaaaataaaaaatacaggcaggttcttgcaacatgtatttcctgtgtaccagaccttgtgttagtccttccttcatgcattcaacatgtattaaacacctactgtgtgccaggcctgatgcctgtaatttattcatacattatttattcaatccaagcaggatttatggagtgcttaggtaatgctaagctctgagcttgtcattcaatgcaatgtatgtttatcaagcagctactgtatacagctgtcccaatcagtgcctgggtacacagtagttcaataaatgcctgaatgaatgaacgccctcaaggagcccccagtcttcctgctaacatggcctgtaaataaataactctaacacacaaagataaggctagaaggtgggtgtgtttagagtgcagagggccccttatcacctgtactgccaggtacctttctcagggccatatcttctcacctgggaaccagctctttgaaggtgaggccaggctggcccctagctcacctgcctgtccatccccgggcacccatggaggactttgcctgcagctcccactcaatcattgcctgctgtctaattacggccgcttccgtgaccgcagttgttccttccttcatacaccaggcaagagggcagcagctgagttctaaccctggagctctggcaaagggcctgcacttctgacaggtgccctccctcagatttggcctgattttccatctatgaaatgaaagatttgattctcctcctccttgacttcttaacaccttgcctggactcctataatggcaagttaattttgaaaaattatagaggtaatccatgatctaattttgcaaaaacccagacaatgcatagaaatcattgaagatgggccaggcgtggtgtctcacgcctgtaatcccagcactttgggaggccaaggcgggcggatcacctgaggtcaggagttcgagaccagcctggccaacatggcgaaaccctgtctccactaaaaatacaaaaattagccaggcatggtggtgcatgctagtaatcccagctactcaggaggctgaggcaggggagtcgcttgaacccaggaagcggaggttgcagtgagccgagatcatgccgttgcactccagcctgggcaacagagtgagacttcatctctaaaaaaataaataaataaataaataaaagaaatcattgaagactcaaatcttcactagagtcacaaagctttgcacaatccaaaactttccttccttccttcttttttttatttatttatttattttattttatttttttgagatggagtttcgctcttgttgcccaggctggagtacaatggtgtgatctcggctcactgcaacctccacctcccaggttcaagcgattctcctgcctcagcctcccgagtagctgggattacaggcatatgccaccacacccagccaattttttgcatttttagtagagatggggtttctccatattggtcaggctggtcttgaactcccaacctcaggtgatccgcccacctcggcctccgaaagtgctgggattacagacatgagctatcgcgctcagcctcctcctctttaactgtggtaaaatatattctgcaggttttttttgtgatggttgtttgtttgtttgtttgtttgtttgtttgtttgtttttgagatggagtctcgctctgtcgcccaggctggagtgcagtgacaccatctcggttcactgcaacctcaccttcctggttcaagccattctcctggctcagcctcagagtggctgggactataggcatgcgccgccacaccccactaattgttttgtgttttttgtagagacaaggtgtctccatgttgtccaggctggtctctaactcctaggctcaagagatctgcccacctcagcctcccaaggtgctgggattataggcttgagcaaccatgcctggcctcctttttttgtgggggagacaggatcttgctgtgttgcttaagctggagtgcagtagtgcaatcatagctcactgcagcctcaacctcctgggcttaagcaagcctcttgtctcagcctctcgagtagctggggctataggtggacaccaccacacccagctaatttttaatttttttatagagatgggggtctcaccatgttgcccaggctggtctcaaactcctgtctcaagtgctccacctacctccccaaagtgctgggattacagggcgggagccactgcgtctgactgtatattaccttttattgtggtcttatcatcagttagtttagaacatttacaggatgaactttaataatgtattgattattttggcaagaaaatggctaataatatttgccgtagtaaaactgagtcattagattactttctggagttacttactgccaagcaaagtaataaagactgttccactgtgggaaggtgtgaaaataaataacattacgcgattctgagcttaaaaataggaaataaacttatatgtacggttttaaaaataataatataggggccgggagcggtggctcatgcctgtaatcccagcactttgggaggccgaggcaggttgatccaaggtcaggagatcgagaccatcctggccagcatggtgaaaccttgtctctaccaaaaatacaaaacttagccaggcatggtggcacacacctgtagtcccagctacttgggaggctgagacaggagaatcgcttgaacccaggaggcttgcagtgagccaagattgcaccactgcactccagcctgggcgatagagggagactccatcccaaaaaaaaaaaaaataatatatatatatatatatatatatatacacacacacacacacacacacaattattactatcgacttaagagagaaaacattaacagtttcatagcagtgctattgacaacaaccacaaaggtgaaaatgacccaaatgtccatcagtagataaatggaaaaataaaacatggtatatggaatataatataatatggaatatgattcagacttaaaaacaaaggaaattctgacacatgctgcaacaagataaaccttgaagacatgctaagtgaaataagccagacatgaatggacaaatatggtatgattccattaatatgaagtacctagagtatttcaattcatggaaacagaaaacagaatgatgggcccaagggctgggcgtggaggcttatgcccgtaattccagcactttgagaggccaaggcgggtggatcatctgaggtcgggagttcgagaccagtctgaccaacatggagaaaccctatctcatggccgggtgtggtggctcatgcctgtaatcccagcagtttgggaggccgaggcgggctgatcacctgaggtcgggagtttgagaccagcctgacctgcagggagaaaccctgtctctactaaaaatacaaaattagccaggcgtggtggcacatgcctgtaatcctacctactagggaggctgaggcaggagaatcgcttgaacccgggaggcggaggttgcggtgagccgagatcgtgccattgcactccagcctgggcaacaagagcgaaaccccatctcaaaacaaaaaaaagagagaaaaagaaaacccatctctactaaaaatacaaaattagctgggcgtggtggcgcatgcctgtaatcccagttactggggaggctaaggtaggagaattgcttgaacccgggcagcggaggttatgatgagccaagattgtgccattacactccagcctggacaacaagagcgaaactccatctcaaaaaaaaaaaaaaaaaagagtcaggcgtggtggcgcgtagctgtaatcccagatacttgggagggtgagacagacgaatcacttgaacgtgggaggcagaggttgcagtgagccgagactgagcgactgcatgtcagcctgggcgacagagtgaaactctatctcaaaaaaaaaaaaaaaagaaagaaagaaagaaaagagagaaagaaaacagaatagaatatagaatagatataatggtggtggccaggggctggatggagggggatattctggggagttaggtttaatggggacagactttttgtttgagaggatggaaaagttctagacgtggatggtggtgatggttgtacaacagtgtgaatatgcttaatgtcatggagctgtacacttaaaaattgttacaatgataaattttatgttacatatattttgtcacaattaaaaaaagcagcagtactcactcactttggcagcacgtatactgaaattgaaaccgcacatagcactttagcatggcccttgcgcgagaatgacaagcagattcatgaggctgagacattccatgttaaaacagcagcaacagaagcccagcaatcccagtgtctctttgaaattctccatggccgggcgtggtggctcacgcctgtaatcccagcactttgggaggccaaggtgggtggatcacgaggtcaggagatccagatcatcctagctaacatggtgaaaccccatctctactaaaaatacaaaaaaaatttagccgggtgtggtggcgggcgcctgtagtcccagctactcaggaagctgaggcaggggaatggagtgaacccaggaggcggagcttgcagtgagccgagatggcaccactgcactccagcctgggcaacagagtgagaccccatctcaaaaaaaaaaaaaaaaagaaattctccacggacccatcccagtctcttcctcctaaccccctgaaagtactagtatatttttgtatttatcgcttattgtaccattgcagggtttttttgtttgtttatttgtttttgagatggagtcttgctctgtcacccaggctggagtgcagtggcgcgatctcagctcactgcaacctctgcctgccgggttcaagtgattctcctgcctcagcctcccgagtagctgggattacaggtgcccgccaccatgcctggctaatttttgtattttttggtagatatggagtttcaccatcttggccaggctggtcttgaactccaggctggtcttgaactcctgacttcgtgatccacccgcctcggcctcccaaagtgctgggattataggcatgagccagcacgcccggcctttttttaaattttttttcgagacagagtcccactctatcacccaccctggagtgcggtagcgcagtctcgtttactgcaatcccctcctcccaagttcaagggattctcctgcctcagcctcccaagtagctggcattacagctgcgcaccaccacacccacctaatttttgtattttttagtagagacggggttttgccatgttagcccagctggtcccaaactcctgaccgcctgcctcagcctctcaaagtgctgggatcacaggcgtgagccaccgtgcgctggcctagtttttatatatattgttttctttggcatgttctttttgttttgaatcattcacttgtttcctttttatttttagagatgggggtctccctctctcacccaggctggagtgcactgatgcaatcacagctcactgcagcctcaaactcctaggctcaagggatcctccttccttagcctccccagtagctaggaccacaggtgcaagccagcatgcctggccaattttttacttttattttttgtagagacagggtttcgccatgttgcccaggctggtcttgaactcctggcctcaaaatatctccctgcctctgcctcccaaagtgctgggtttacaggtgtgagccaatgcacccagccatttcttcttttttgatttaaaaatatttaattgataatggccaggcgtggtggctcacatctgtaatcccagcactttgggaagccaaggcgggtggatcatgaggtcaggagttcaagaccagcctggccaacatggtgaaaccctgtctctactaaaaatacaaaaattagccgggcgtggtggtgcgcgcctgtaatcccagctactcaggaggctgaggcaggagaatagctcgaacccgggaggcagaggttgccgtgagccgagatcgtgccattgcactccagcctgagacagagcaagactctatctcaaaaaaaaattttaactgataaattgaattgtatatattcaagatgtacagtgtgatgagttgaaatccattgtgtcctgattaccatggtcatattgattaacgcatatccatcactcccatagtttccattttacttgtcctgtctttgaattttatgtaaatagacttgatagtatacatatacaattgtccgaatgttagtgtccccccaaaattcatgtgttggaacctaatacccaatgcaatagtattaagagataggtcctttgggaggtgattaagttataaggttctgcgtgtgtgaagggattagtgccctcataaaagcagctcgagggggctatgtgctgtggctcatgcctgtagtcccagaactttgggaggctgaggtaggcagattgcttgagcccaggagttcgagactggcctgggcaaacatggggagaccccgtctcactacacaatattagccggttgtggtggtgcattcctgtggtcccagctacccaggatgctgaggtggaaggatcacctgagcctgggagaatgaggctgtagtgagctgtgattgtaccactacacttcagcctgggcgacagagtgagatcctgtctcaacaacagcaacaaccaaataaataaataaaagaggtccccagcctggccaacatggcgaaatcctgtgtctactaaaaatacaaaaatgtgctaggtgtggtggtgcatgcctgtgatccccaagggaggagaatcacttaaatccagaagctggagcttgcagtgagctgagatcatgccattgcactccagcttggacgacagaactgaccttgtctggaaaagaaaaaaagagacccgagtggagaagctagcccctttccaccatggaagatacagcaacagggtgccatttttgaagtaaactaagtcctcaccagagaccgagtctgctggtgacttgaccttgaacttcccagcccccagaactatgaccaatacattgctgttgttttatttatttatttattttattttatctgagacagagtttcactctctcacccaggctggagtgcagtggcttgatctcagctcaatgcaacctctgcctcccaggttcaagcaattttcctgtcccagcctcccgagtagctgggattacaggtgcccaccaccatgcccagctaattttttgtatttttagtagagacagggtttcaacatttttgccaggctggtcttgaactcctgacctcaggtgatccacccacctcagcctcccaaagtgctgggattacaagcgtgagccaccatgcccagcctagttttattatttgtagagatggggtctggccatgttacccaggctgatctggaactcctgggctcaagtgatcctcctgacttgacctccaaatgtgctgggattataggcacctggcttgaaattttgtttgtttgttttgttttgttttgagatgggggcttactctgtcacccaggctggagagcagtggtgcgatctccacccgctgcaacctctgcctcccaggtttaagcgattctgatgcctcagcctcctgagtagttgggattacagtcatgcgtcacaacacactgcctggctaatttttgtatttttggtagtgatggggtttcgccatgttggccaggctggtcttgaactcctaacctcaggtgatctacccgccttggcctcccaaagtgctgacattgcaaacgtgagccaccacactggagagataggcaggaactagatcacatcagtctttcccatgctggggaaggcgcttggatttcatcctaactcagattctcagatgcatggtcactggagggctgtacacagtgtggcaggctgagtaaatgtcccccagggatgtccatgtcttactgtctggcacctgtgaatatgtgaccgtatatggatgaagggactttgcggatgtgatggagtgaaggattttaagattgatcctggattacctgggtgggcccagtgtaaccacaggtgtcctcaggagaggtagaggaggtcagagaagcgatgtgacaatggaagcagagtctggagaggcgtgacctgctttagaaacagaaggggctgcacactaaggaatgcaggcagccagcagaagggaaaaagcaagaaatggattttcccattatctcattggagcctccaaaggaaccagccctgctgtcaccttaattttggcccctgatgattcattttgaacttcttacctctagaactaagataatgaatttgtgtgtgtgtgtgtgtgtgtgtgtttgtgtgtgtgttttgagatggagtctcactctgtctcgcccaggctggagtgcagtgatgtgatctcggctcactgcaagctccgcctcccgggttcaagcaattctcctgcctcagcctcccgagtcgctgggattacaggcacccgccaccacgcctggttaatttttgcttttttttttttttttttttttggagacaaagactcggtcttgtcccccaggctggagtgtgatggcgcaatctcggctcactgcaacctctgcctcctgggttcaagagattctcatgcctcagccacctgagtagctgggattacaggagactgccaccacgcccggctaatttttgaaattttagtagagatggggtttcaccatgttggccaggctggtctcgaactcttgacctcaggtgatctgcccacctcggcttcccaaagtgctgggattacaggcatgaaccgccgcgcccggccaatttatggtaatttcttacagcagcaatgggaaactaatacaagcagagaaaggacatgatcagatttatgtttcaaaaactatttcttgttgctattacagaatgggctttagcgggatcagcagaagcagagagcccgggtgggtaactgcagacgtgtgggtgggcaacaatggcagcccggatgtgatggaatggttgctgctggacggaagagctcagatctcaagaaaatgctaggggaggccgggcgcagtggctcacgcctgtaatcccagcaccttgggaggccgaggcaggtggatcacgaggtcaggagatcgagaccatcctggctaacaaggtgaaaccccgtctctactaaaaaatacaaaaaaaaattagctgggcgtggtggcgggcgcctgtagtcccagctactcaggaggctgaggcaggagaatggtgtgaacccgggaggcggagcttgcagtgagccgagatcgcgccactgcactccagcctgggcgacggagcgagactccgtttcaaaaaaaaaaagaaaaagaaaaagaaaatgctaggggctagaactgactgggtttattgatgaattggatattgtggggaagggtgcagtctaggaaagtgactgcagcgtgactcctggggtgatcagaactacaatttctccagcttctctggggcctcacagagccacactgtgtttgacaggctgttcccaggatgcgggtgtttttatgtgattaggctggaactgttctctgggaaataaatgccctcagtctaaagggcttttacgagccagggcaggggattaaaggcggccgggctgtggagaaatcccagggttaggagacagctgagcatggagacttttaatctaaaccttaaacgtttttattttccactccttgaactcagggtgtcactttaaatacatgtttggcaacatcagctgttctatttttctttttaattatacaaagcgagatcctggaaatggctcagccccaggctgggggaggcaagggaagtttggcaatggtaggaggcagagaaaccaactaaaaatctagtggatttgaaatccttttgagggagcacagaggccaaacttcgagccaccctgaacttggtaagagctgccctgactcaggatttctaaaaggagaccgacagactcaccagcttagaatggagagaggacagatggacacgtctccaagacaaaaatatcaccagccctagcactgttcctgtacctcgaatttattcccaggccctgtcctgttagctcagctcttttcatttcttttcttctttctttctctttctttcttttctttctttccctttcttcttgctctttctttcttttcttttctttctttctctttttctcatttcttttcctctttctctctctctctttttttttttttattagaggcagggttttactatgttgcccaggctggtcttgaactcctgggctcaagcgatcctcctgcctcagcctcccaaagtactgggattacaggtgtgagccactgtgcctggccctgttacctcctttcatcctctgagcagaaaacagctaactaggttgttcatgacatctatggggtactccctactgtgtgccagctgctgggctcacctgcttacacgtgttacactgttgtaggcagtcagctattctataagtaggtgctattattatccccgttttctagatggggaatctggaatctccagagagaacggatggaacccccacaccccagtgttgggcctgcacttctttgcccttggacccctcccttgcctttctctagccttgctccagatcacagggtggccaacctttggcagggtggcttcctgcacagatggaccaactggaggcagtgccaggagcctggtgggtgggaggagcctggtggtgggaggagcctggtgggtgggaggagccaggtggtgggaggaggggaatccagggtgttcctacccctctgcctcaggtgggatctccagctccacctcccaccagacaggtccactgtggctctggcttctgcctgaggactccagtccctcttcggtaccagaaacaccaccccctgcctctgtccctccaaccccagggtgatagcagtcctcctgctggggctaacctgggggctgcccttcccctgcctgccttttcctgtaagtcttccattacccctggagcaattctcaggataaatgctcttagtgtggtaactgtttgtcctggtcagattctcacagatatactaccttcactaaagacttacagatcagtcaattcattcaatctggaaggcagcaatcctctttctctgagcacagcacatcccatgagagggaaggttggagccaccatttactgagcgctgtgctaattgccagacaacttttctttcatgaaacactcataacaacctctttgccaagtcccatcaatattcccctttcacagatggggctcagagaggctggggtgcttgcccaaagtggcacagttacaaactgacaaaactgggattcaaatccaagagtgcctagctccaaactctgcataatgatctaatctcatctgaacacacttgcagccccattagaattcaacttccttggctggctgaagaccccaccccaaatcactccagcctccttgtattcttttcttcttctttttttttttttttttttgagatggagtctcactctgttgcccaggctggagtgcagtggcgcgatcttggctcactgcaagctctgcctcccaggttcacgccattctcctgcctcagcctcccgagtagctgggactacaggcatccgccaccacgcccagctaattttttgtatttttagtagagacagggtttcaccatgttagccaggatggtctcgatctcctgacctcgtgatccgcctgccttggcctcccaaagtcctggcgtgagccaccgctcctgacctgttttttgttttttgtttttttttttttgagacagagtctcactctgtcacccaggctggagtacaatggcctgatctcagctcactgcaacccccgcctcctgggttcaaacgattcttccacctcagcctcccgagtagctgggactacaggcgtacaccaccattcccagctaacttttgtattttttagtagagacagggtttcgccatgttgcccaggctggtctcctgaccgcaggtgatccgcccacctcggcctcccaaagtgctgggattacaggcgtgagccatcacgcccggcccccccaaaaaaatttttttaagtcccacaaactgggtggcttaggagagaaacttattgtctcacagttctggaggccagaagtccaaaatcaagatgtcagcaaggccacggtccctcggaaggaaccagggaaggacatgccccaggccctgctgctcgagctacctggcttgtagacggccgtcttcttgtgtccattcatgtggtcttccctctgtacatgtctgtctgtgtctaaatttcctcctttgtttttgttttttgttttttgttttttttttctgacagagtttcgctcttgttgcccaggctggcgtgcagtggtgcgatcttggctcactacaacctcctcctcccgggttcaagccattctcctaccttagcctcccgagtaactgggattacaggcacatgccaccacgcccggctaattttgtatttttagtagagataagttttctccatgttggtcaggctggtctccaactcctgacctcaggtgatccgcccgcctcggcctcccaaaaggctgggattacaggcgtgagccacggtgcctggctgtttttgtcttttttgagacagggtctcacactgttgccaaggctggagtgcagtgtcacgctcacagctcagtgcagctttgacctcatgggatcaagcaatgctcctgcctcagcctcactagtagcgggaaccccagacaagagccactgctccttgcaaatttcacacttctcctttttataaggatattagtaatattggaagagggctgtatgagtctgttctgtcattgctataaagaaataccaagctgggcacggtgactcacacctgtaattccagcactttgggaggcccaggccagcagatcacctgaggtcaggagttcgggacaagtctggccaaaatggggaaaccctgtctctactaaaactacaaaacattagtcggacgttgtggcaggcgcctataatcccagctactcgggaggctgaggcaggagaatcgcttgaacccaggaggtggaggttgcagtgaggcgagattgcaccactgcactccagcctgggcaacaagcacaaaattccatttagaaaaaaaaaaaaaaaaatgaaaagaaatacctgaggctgggcaccatggttcacgcctgtaatcccagcattttgggaggccgaggcggacagatcatttgaggccaggagttcgagaccagcctggccaacatggcgaaaccccctctctactaaaaatacaaaaattagccgggcgtggtgatgcactcctgtaatcccagctactcgggaggctgaggcaggagaattgcttgaaccagggaggtggaggttgcagtgagccaacattgagccactgcactccagcctggaagacagagcgagactccatctcaaaaaaaaaaaaaaaaaaaatacctaggccgggcacagtggctcacgcctgtactcccagcactttgggaggctgaggcaggtggatcacgaggtcagcagttcaacctgccggtccaagatggtgaatcctcatctctactaaaaatacaaaaattagctgggcgtggtggcaggcacctgtaatcccagctactcgggaggcggaggcagagaattgcttgcacccagaggcggaagttaaagtgagcccagatcgcgccactgcactccagcctgggcaacagagtgagactctgtctcgaaaaaaaaaaagaaaaagaaatacctaagactgagtaatttataaagaaaagaggatgaatacaaaaaaatcagctgggcgtggtggcagtcgcctgtagtcccagctactccggaggctgaagcaggagaatggccgtgaacccgggaggtggagcttgcagtgagctgatattgcgccactgcactccagcctgggcgacagagcgagactccgtcaaaaaaaaaaaaagaaagaaagaaagaaagaaaagaggattaattggctcttggttcggaaggttgtgcaggaaacattgcagcatctgcttctggggaggcctcaagaagcttccaatcatggcggaaggcaaaggaggaaccagcccttcacatggcgggagcaggaggaagagaggggcggggaggtgctatacacttttaaacaaccagatctcactcactgtcatagcaccaagaggatggtgctaaaccattcatgagaaactggccccgtgatccaatcacctctcaccaggctccatctccaacactggggattacaattgaacatgagatttgggtggggacacagatccaaacgaaataagtgcccaccctatgacctcattttaatttgattacctctgtaaaggctgtatttccaaatgaggtcacattcttcgtttttttttgtttgtggtttttttttttttttttagagagtttcactctttcgcccaggctggagtgcaatggcacaatctcagctgactgcaacctctgccttccagtgtcaagagattctcctgcctcagcctcccaagtagctaggattacaacagtcgcttgccttcacacccagctaatttttgtatttttagtagagatcgggtttcaccatgttggccaggctggtctccaactcctgacctcctgatccaccctcctcggcctcccaaagtgctgggattacagatgtgagccaccgtgcctggccacagtcttcttcttttgtttttttttgagacggagtctcgctctgtcacccaggctggagtgcagtggcgtgatctcggctcactgcaagttctgccttccaggttcacgccattctcctgcctcagcctcccgagtagctgggactacaagcacctgccaccatgcctggctaattttttgtatttttttcattagagacgggggtttcaccgtgttagccaggatggtctcgatctcctgacctcgtgatccgcctgcctcggcctcccaaagtgctgggattacaggcgtgagccatcgtgcccagccggccacattcttctttttgaggtggagtttcactcttgtctgccgggctgcagtgcaatggcgcaatcttggctcactgcaacctccacctcctggctactttttgtattttcagtagagatggggtttcaccatgtttgccaggctggtcttgaactcctgacctcaggtgatccgcctgcctcagtctcccaaggtgttgaaattacaggcgtgagccactgcgcccagccccaaatgaggtcacattctgaggtactgaggtttaggactgcaatgtcttttttggggggacacagtttaacctttaagagtccaacctgatctcatgtgtctgatctctgcagtgtggcctgctttgctaatagaggatgtcagaccgtccaggaaaatgatcctgctgacacccaccaagttaagggatatagtgattctccaaggcatatgtgtattgacaaaatttaccccaacattctgtacagcaaattcagttttgaaaaaggagaactagactaggcgtggtggctcatgcctgtaatccagcactttgggaggctgaggtgggtggatcacctgaggtcacgagttcaagaccagcctgaccaacatgtagaaacctcgtctctactaaaaatgtaaaaattacccgggcgtggtggtgcatgcctgtaatcccagctactcgggaggctgaggcaggagaatcacttgaatccaggaggtggaggttgcagtgagctgagatcatgccattgcgctccagcctgggcaacaagagcaaaactccgtctcaaaaaaaaaaaaaaaaaaagaaagaaagaaaagaaaagaaaaagagaactaggctgggtaggttagctcacgcctgtaatcccagcattttgggaggccatggcaggaggatcgcttgtgtccaggagttttgagactggcctaggcaacatattagatcccatctttataaataataaaaaattagccaggtgtggtggtgtgtgcatgtagtctcagctacttggcaggctgaggtgggagggtcacttgaacccaggaggtcaaggctgcagtgagccataatcgtgcctctggactccagtctaggcaacagaatgaaacttcatctcaaaacaacaacaacaaaaaagaagctggtcacagtggctctagcctttaatcccagcactttgggaggctgaggcaagcggatcgcttgagcccaggagttcaagaccagcctgggcaacgtggcaagaccccctgtctctacaaaaaaaaaaaaaaaaaaaaaaacacaaaaattagccagatgtggtgatgcaggcctgtagccctagctacttgggaggctgagctgagaggatcacttgcacccgggaggcagaggttatagtgggctgagattgtgccactgcactccagtctgggcaagagaccaagacccctgtgaaaaaaaaagaaggaaggaaggaagggaggaagggagggagggaacaagttgaaggtcttacctctacctgactttaagacttattttgaggccgggcgtggtgactcatgtctgtaatctcagcactttgggaggctgaggtgggtgaataacctgaggtcaggagttccagaccagcctggccaacacggcgaatccctgtttctactaaaaatacaaaaattagctggatgtggcagcaggcacctgtaatcctagctactcgggaggctgaagcaggagaatcgcttgaacccgggagccgaggttgcagtgagcccagaacgtgccattgcactccagcctgggcaatagagagagactctgtcttaaaaaagagacttttttttttgagacagggtcttgctctgtcacccaggctgaagtgtagtggccccgtctcggctcactgcaacctccacctcccaggttcaagcaattctcctgcctcagcctcccgagtagctgggactacaggtgcgtgccaccacatccggctaatttttgtatgtttagtagagacaggatttcactgcgttgaccaggctggtctcaaactcctaacctcgtgatccgcccgcctcggcctcccaaagtgctgggattacaggcgtgaaccaccatgcccggccgagacttttttttttttaattttgaggaatttatggggttttttttccccaagacttattttatttttatttatttatttattaatttatttacttttttttttttttttgagatggagtctcactctgtcacccaggctagagtgcaatggcgcgatctcagctcactacaagctctgcctcccgggttcatgccattctcctgcctcagcctccagagtagctgggactacaggcgcctgccaccacgcctggctaattttttgtatttttagtagagatgaggtttcaccgtgttagccaggatggtctcgatctcctgaccttgtgatcggcccgcctcggcctcccaaagtgctgggattacaggcatgagccactgcgcccggccaatttatttacttttgagacggagtcttactctgtcgcccagtatggagtgcagtggcgtgacctcggctcactgctacctccgtctcccgggttcaagggttctcctgccttagcctcccaaatagctgggactacaggtgcgggccaccatgcccagctaatttttgtatttttagtagagatgaggtttcaccatgttggtcagactggtctcaaactcctgacctcaagtgatccgcccaccttggcccccccaaagtactggggttacaggtgtgagccaccacgccctgcccccaagatttactttaaacctagagtaatcaacacagtatctattggcatgagagtagacaaatagatccacagactagaatagagagttcagaaacagtctgtcatgtatagtagtcacgtgatttttgacaaaaatgtccatgcatttcagtggggaaagcttgcttcactcaaaaatctgtatttgattgatgggctataggctcagctacttgagaagctgaggcaggagtgagaatcacttgagtcatgaatttgaggctgtagtgtgcaatgatcacacctatgaagagccactgcactccagcctgggcaacagagcaagaccttgtctctaagaaaaaaacagaaatagaaaaaaatcagtatgacacggatcacagacctaaacatacaagcaaaaacacttttatttttggcggggtgggtggagggtagagacagggttccactatgttgcccaggctggtctcaaacctcctggtctcaagtgatcctctcgacctggcctccccaggtgctgagattataggcatgagccaccatgcctggcctaaaacttttaaaagaaaacacaggtgaatatcttcgtgaccttgtggttggcaaagatttctttttttctttttttttttttttaaatagagtcttactctatggcccaggctggagtgtaatggtgtgatctcagctcactgcaacctccgcctcttgggttcaagtgattcttgtgcctcagcctcttgaatagctgggattacaggcatgtgccaccatgcccggataatttttgtatttttagtagagatgcagtttcaccatgctggccaggctggtctcgaactcctgacctcaggtgatccgtccacctcagcctcccaaaatgctgggattacaggcatgagtcaccgcgtcgtcagcaaagatttcttatccaggccagaaaaagttctcacttttaaaggcatgggcacaaactcagacccaacaaagacttctgaccagtgagggggtaggagtggagggaggaagaatgagcactataagcccagaaactggagagcaaggagacagagttcacactcccctggcatagtctaagctccagagtcagagaaccattctgagaccaacaccagggctcaccaacccgaccccattctgcattttgtaaccagccccctgacagtgtctcacttggtttccagcctccttcaccccaattctttgaccatgaaaaccttcaagatcactcacaaaagctgcctcccagatgtcttgtggagctggcaccagggctggctggcagagggatttcatgcattctgagtcaggacacaaggaaggactgacagggaagctgcccccagctccatacaaggacaacttttgaagtatcagacaaccaatcagacatgctcagcttctagaaggagtgagcttcccatcgccaggggtatgaaaacattgttctagccgggcttggtggtgcatgcctgtggagcccaggagtccgaatgcagcctgggtaacacaatgagacccctgtctccccaccaccaaaaaaaaaaaaaaaactgtggggaaaagttagtaaccatggacccctctaggatcctggggcaggaggcagggatggtccagacaccccacccaagtttttattacttgtgatctgtgcagatggggaaattcaaaccacacagaatattaggagaggggtgaggagcgtgacatagtagctggggaaaggaaaccctcaccaccccaaagccccctctgagtcaccactcccagatccttttgttcctctaaaatcagcaaatctctcctcccagcttccggcctcttccccctccagacctcaggccctcttagctgctaaatgttgtgtgttcctagtcctgaactgggggtttctgggaaccaaggacattgcattgttttgttcattgaacctgtttccattggcagtagccagtgggaggccctggggtgatagcgacgtgattgattgattgattgattttgagagactgagtctccctctgtcgcccaggctggagttcagtggcaggatctcggctcactgcaactttcgcctcctggattcaagcaattcttctgcctcagcctccggagtagctgggattacaggcctggccaccacacccaactaattttttcttttttgagatggagtctagctctgtcacccaggctggagtgcagtggtgtgatctcggctcattgcaacctctgcctcccgggttcaagcaattttcctgcctcagcctcccaagtagctgggactacaggtgtgtgcccccacgctcgactaatttttgtatttttagtagagacggggtttcaccatgttggccagactggtctcaaactccagacctcgtgatctgcccgccttggactcctaaagtgctgggattacaggtgtgagccaccgcgcccggctaattttttcttattttgacacagggtcttactctgtcgcccaggctacagtgcagtggcgtgatctctgctcactccaacctctgcctgccgggttcaagcacttcttctgcctcagcctcccaagtagctgggattataggcatgcaccaccaagcccggctaatttttgtatttttagaagagatgggcttttaccatgttgaccaggctggtcttgaactcctgacctcaggtgatccacctgcctcagcctccgaaagggctggaattacaggcgtgagtcaccgtgcccggcctaatttttgtatttttaatggagacggagtttcgccatgttggccaggctggtctcaaactcctgaccttaagtgatctaccgcctcggcctcccaaagtgttggaattataagcatgagccaccacacctggcccatgattcatttatttatttagttgttttttctttgagacaggatctcgctttattgcctaggttggaggacagtggtgtgatcaccgcagcttcattccaagaagctgggagtacaggctcacgccaccacatctggtttatttatttatttattgagacagagtattgcttttattgcccaggctggagtgcaatggcataatctcatctcatcgcaaactccacctcccgggttcaggcggttctcctgccttagcctcccaagtagctgggattacaggcatgcgccaccacaccagctaatttttgtatttttagtagagacgaggtttctccatgttggtcaggctggtcttgaactcctgtcctctcaggtgatccgcccacctcagcctcccaaagtgctgggattacaggtgtgagccaccgtgcctagtcttatttatttatttatttggagacagagtctcgctctgtcgcccaggctggagtgcagtggcaccatcttggcttactgcaagctccgcctcccgggttcacgccattctcctgcctcagcctcccaagtagctgggactacaggcgcccgccaccacacccagctaatttttttgtatttttagtagagacggggtttcaccgtgttagccaggatggtctcaatctcctgacctcgcgatccgccggtttcagcctcccaaagtgctgggattataggtgtgagccaccgcgcctggcctttttttttttttttttttttaagtagagatgtgctactgcactccagcctgggtgactattgagaccctgtctcaataaataaataaataaataaataaataaataaataaataaataaggccaggcgcagtggctcacatctgtaatcccagcactctgggaggctgaggcaggcagatcatctgagttcaggagttcgagactagcctggacaacatggtgaaaccctgtctctactaaaaatataaaaaattagctgggtgtgctggtgggcacctgtaatcctagttactcaggaggctgaggcagaagaatcgcttgaacctaggaattggaggttgcagtgagctgagattgagccactacgctctagcctgggggacagagcgagactccatcttaaaaataataaaaataaaaataaaataaaatgtttaaatacgttgggcacaatggtttacacctgtaatcccagcactttgggaggctgagatgggtggatcacttgaggtcaggagttcgagaccagcctggccaacatggtgaaaccctgactctactaaaaatacaaaaaattagctgggtgtggtgatggacacctgtaatcccagttatttaggaggctgagccagaataatcggttgaacccaggaggcggaggttgcagtgagccgagatcgagccactgcactgcagcctggggaacagagagagactctgtcctaaaataataataaaataaaataaaatgtttgaataggctggacacagtggctcatgcttgtaattccagaactttgggaggcccagatgggaggatcccttgagcccaggagtttgagaccagcatgctcaacatggggagacgctgtcactattattttttattggagacggagtctccctctgtcgcccaggcttgagtgcagcagcctgatctcagctcactgcaacctctgcctcctgcgttcaagcaattatcctacctcagcttccccagttgctaggattataggcgtgccccatcatacctggctaattttttttgtatttttagtagagatgggggtttcaccatgttagccaggctggtctcgaactcctgaccttaggtgatccacccgcttgggccttccaaagtgttgggataacaggtgtgagccaccgcgcctggccacacttttttttttttttaatgcttaaacagatgaacaggtcaactaaataagctagatgatttcagatgctgtagtgatcacagctataaagggaagaaatcaggatgaggtgacagattgtgacaccggggctacttcaatagagggaaggtctctcaggaggtgctatttgagctgagacacaaatgttgagtgaaaggggtaagaagtacaaaggcgggccatggcgcggtgcgtcacgcctgtaatcccagcactttgggaggctgaggcgggtggatcaccaggtcaagagatcgagaccatcctggtccatatggtgaaaccccgtctctactaaaaataccaaaaatagctgggcgtggtggcgcgcgcctgtagtcccagctactcggcaggctgaagcaggagaattgcttgaacccgggaggcggaggttgcagtgagccgagatcgctccactgcactccagcctggtgacagagtgagactccgtctcaaaaaaaaaaaaaaaaaagtacaaaggccaccagggaaacaaagccagtagggtgagatttcaaattccaaattccccaatttttttttttttttttgagagggactctcgctctgtcgctcaggctggagtgcagtggctcgatctcggctcactgcaagctccgcctcccaagttcaaacgattctcctgcctcagcctccctggtaactgggactagggagtcacctgactaatttttttagtagagacagggtttcaccatgttggctaggctggtctggaactactgaccttgtgatctgcctgcctcggcctcccaaagtgttgggattataggcgtgagtcaccgcgcccggcccccatctttattttaaaaaagccaagaaaaggacaaaaaaccaaacaccgcatgttctcactcataggtgggaattgaacaatgagaacacatggacacaggaaggggaacatcacactccggggactgttgtggggtggggaaaggggggagggatagcattaggagatatacctaatgctaaatgacgagttaatgggtgcagcacaccaacatggcacatgtatacatatgtaactaacctgcacattgtgcacatgtaccctaaaacttaaagtataataataataaaattaaattttaaaaaaaaagccaagaaagggctctgcgggaaccagagacttcttgggggcagaggttttcatcccacaacggttgggtgggggcgcgccctctagtggtgacaagagttacgtttcaccccgccaaccggcaatgtcacatgctcgtcactcattcctgtctctcccatagttattcattcaacaaaaaattactgaggaccgattgtatgccgaggaatattctagatgctgagggtagagctgtgaagctgatagtaaaggtccctgccccggagtttacctcctaggagacattccctagcacagtcctgcatacacccccacctcacctctgctccccaaaattggccttccaacgctccaacagccaaatatccctgcttaaaggtcttggcatgagcagttccctcgccgtcctggaaagctagatcctgtctgtttgcatggcgggctctggtctcagcttaaatgtgacctaagtcagaaaggtgtgatctcacttatttcctgcccctctccacactccactaaaatataatattcgggccgggcatggaggctcatgcctgtaatcgcagcactttgtgaggccgaggcgggcggatctcctgacgtcaggagttccaggctagcctggccaacatggcaaaaccccgtctctactaaaaatacaaaaattagccaggtgtggtggcgggcgcctgtaatcccagctacgcagcaggctgaggcaggagaatcgcttgaaccgggaggtggaggttgcagtgagccaagatcgcgccattgcactcctccagcctgggcgacaagagcaaaaccctgtctcaaaaaaaaaaaaaaaaaaaaaatatatatatatatatatatatatatatatatacacacacacacacatatatatacatatatatgtgtatatatgtgtgtgtgtatatatatatataaaatattcaatgaaggttatcattttcctggaatccctcagcgtagcacgtaatagttgctcagaagacacttgctgaataggtttgaagattcactggtttaaaaaatatcaggtggccgggcacggtggctcacgcttgtaatcccagcactctgggaggccgaggcgggcggatcacaaggtcaggagtttgagaccagcctggccaatatggtgaaaccccatctctactaaaaatacaaaaattagcagggcatggtggcgggagcctgtaatcctagctactcgggaggctgaggcaggagaattgcttgaacccagtaataaaaaaataataaatcaggaaccggtcacctcaatacgtatcagatgctggtgaacacaggtggacaaagtggttaacagacaggttttgtccatctcgttcaccaccctacccagcctcagcacctagaccagtgttggcacccagtgggcgccaaataaacactgcttgaactccagacgtcagccgctctttttcctacagaccttgagccaccttgttccaaaggggatatgggcctcaggaggcgcccagaggtgacctcaggcggcccgacccaggagtccaagctccaggagcagggccacgggagcagctgcggagaggggcggcgccaggagccggagcgggcagccgggcgcttccaggaaaagtggcgggcggcggcgcgccagggaccgtgggcggtgccgtcggagcgggcgggtcacgtgacgcccacaacaacgcccacttcttggtgggcggggcacaggtgggcggggagcatgcaaaacagcccagggcggcggccaatcgcggcgcgcgccgggggtccaggccccggggatccgaggcgccgcccgcgcgcagtctctggtcactgccgcccgggggcttttgccagcggcgccgcgggcctgcgtgctggggcagcgggcacttcttcgacctcgtcctcctcgtcctgtgcggccggccgggtgaggccgggcccgcgtagggggcagtcggcggctgcctccggcggaggtgcctcgcggcgcccgggccggcccgcgcctcggcggcgtgctccatgcatccggagcccgccccgcccccgagccgcagcagtcccgagcttcccccaagcggcggcagcaccaccagcggcagccgccggagccgccgccgcagcggggacggggagcccccgggggccccgccaccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtgagctccggccgccctgccctgccccctctactgtcccggctgctcccgcccgcagccccgacggggctctctgggcgggccgggcgggccagagtgaggggaagagctgggagaaaccgtggtgagcgctgcgcggggggcagaggtgctggacgtgagcactggtgggcgccgagtactccgatctggggtggtgacaagccatatcagagtccgggctcggggtgcttgggtgcctccacatggccatgcctgccccagcaagagtggtagatgcatcccgaagttagctctgtaagaagcccgccccatgtgcgttcaggagcgaaaggagggactcagaagctctaacccagctgggggcgtcaggaggtggggacaaggagaaagaccttgcgctgtcttgtgccccggcacccgaggccggaagttgttagccgtgttcttttatcagctcctagaggggatttcctgacaccccttttccaccgcattttccgagagcctcagcctgggggtttgacaaccagtccccctcagttttttctccagaaacttgccactgagtcactgcgtgtgagggaggagggagggacatgttcccgttgaatgggctctttattttttatttgttccaagacggagtctcgctctgtcgcccaggctggagtgcagtggtgcaatctcggctcactgaaatttccacctcctgagttcaagcgattctcatgcctcagcctcccgagtagctgggattacaggctcgcgccaccacgcccggctaatttttgtgttcttagtagagacggggtttcaccctgttggccaggctgatctttaactctcgacctcgtgatctgccaaatctcggcctcccaaagtgctgggattacaggcgtgagctaccacacccggcccaatgggctctttcttgatctagggcataggcatgctttcctttccccaccatcccaaagagctggagtgcaaagcctctgaggtgtggggtgggtgaggctgcggatgggcctgagaagtggctctggggataccctcaggcttggcatattgaagtttctccgttgcaggggactacataataactacattattaccactcggtgatatcagcctgccatgaatggactctgctccccgcccctcagttctgtagaccagagcactcctgcccccgccaccagtgagctaagcccttggccttgagggagggagtggctgcacccttcgtgttttcttgggaagggagtgtgtctgaaactctccctgctttgccttgcagcagagcctgaattttttaaggcccagagcacgctgggccacagtggaaaggccgggagatggggagtgggaagcagccttccctgctcagttacttgctgcctgtgtgacctcaagcaactcactctccctgtccgggcctcaattcctgaattgatcaaagaaggctgctactcaaaagtttctctgactttgagaataaaattgagggagcccctgaaaggagatcagggagtgtgtttgcataaacagacggcactctcatttttagtttagacaggacggacttctgctctgttaatttcaaatcctaagagaccaggttctgcctgctgcctgaagaatctctaaaagcagctggcatttactgaacactcactcagtgctaggtgccttaactcctgggcttactcaacaaccctctgtggtgattttgtcaaagggctctcccaagaccgcacagccagtctggtggagcgggatttgaacccagctctgctggtgttcagaacttgctcaggcacttgtacccattgcggtgtataggaactctgattgatgtccagccagggatgtgttaggacactaggaaatgagaaatagggcaaaagagtttctttttttttttttttttttttgagacggagtcttgctctgtcacccaggctggagtgcagtggtgcgatcttggctcactgcaagctctgactcctgggttcacgccattctgctgcctcagcctctggagtagctgggactacaggcgcccacaaccacgcctggctaattttttttgtatttttttagtagagacggggtttcaccgtgttagcgaggatcgtctcgatctcctgacctcatgatccacccgcctcggcctcccaaagtgctgggattagaggcgtgagccaccggcgcccggccgcaaaagagtttcttttaagccaaagaaagttttataattggtggtggggagggagctaccagttgtcagcttttccctgcagcgcttggtatttaagataatggttcccccttttcttgtggtcatgagtttatagctgccccaggctccttctcactcctgaggggtgagatccagatgaatgacactttcctgccttctcaagtcccttcttcctttcgaagacaggcctcatggtcaaatgtcacctccttctcttgctaaaaacagtgtggggaagtcaggagactggggcagccagtggcatggagtgggccttggcccacccagtctgtctcttggggacttttattttcccccacctgggtggagaaggcttcctggcactactgatggaatccccaaagttctgcaggcagggaaggaccgaaccccatgatactctccattctgcagttacaagaaggtaactgcatgtgccatgaagttggaaatctataggagggatgggattgggtgcagggtctgaccatgccccactcccacttagacacccagcctgtaagtgagcttgggagaggagagggtgccagcagccccaacgccatggtaacagagtgacaagtttcctggattactctcccttggcttatgagtcagcacaaccaacttcctgcccccagagggcttccttcccagagacctggaaagtcagaggagctgacttgacctttgattgccaagggatggggtcaaccaggtgagattgagggagggtctcccacaaatcctggaggggattgagaggttccccacttcatgggtagaacagcctggccaggtggccgggaagtgttggggatggctggtgggtgacctcaggcaagaggctttacttctttgaagccagttttcttttcttttcttttctttttttttgagacggagtttcctctttgttgcccaggctggagtgcagtagcacaatctcagctcactgcaacctccacctcccaggttcaagcgattctcctgcctcagcctcccgaatagctgggattacaggcacacaccacctcacctggctaatttttgtatttttagtagagacggggtttcaccatgttggccagggtggtctcaaactcctgatctcaggtgatccacccgcctcggcctcccaaagtgctgggattacaggtgtgagccaccatgcctggctgaagccagttttttaatgtgtatggagatggcaggtatcctgctctcccatggactcatatgaaggttcattatgaggccagactgggcccatgaaacacaggaattttcagggtctgatgatgcacaccttgcccggaccctctctgatctgcagatgagattcttgctccacacggcagttctaggaacagcaggcctcaggcgcaccatcctccaaactctaatgagagatacaaagcaagggggcctggatatagcataggatcagcatcccgtagagctccttaagcccagagtactcttgtgggctttgattcaggtcctctgtgcctcagtttatacttcattgagaggcagccagcctcagcctgaccccgtcccctgcaggactggcgagcatatgtggtgatcttgtctgctggggaaggcaggctgtaacattagcctgagctgaagataattgggaaggtcagtctgctgccgtaaacaaaatgctttccatgacctgggatcaattatttttggattatctgcctcctcagttctctgcccgccagcacccttttgttcccaagttctagagctggttcagaagtttcctgtaatttctatgaaaatgagttggaaggaacgactcaagtcataagacactgagcctctgtgacgtgttcagagttcagggaccatgtaaatgagcaagaaattgctattactattcctagtactgatcactgatagtaattattattgctgttgttgattgggctcttgcaataggctagatggtgccccacgtagcatcttgtgtgatcctcctataaaccaggtgagtataggtgatggtcattgagaggaaagacttgcgcatggtcacctggctgggaagctgcagagccctgtggctgtggtgtagcgccattgctgtgggagagaaagcatcagagagtgtaggggaacagagctgagaagctcaagcagggtaaggtcagaacagacacttgagcaagacaacttggttcaaatcccagctctacctcttataagggcaagaccttaggcaagtcatttaacctttctgggcctcagtttcttcatctttaaaatggagtacggccgggcgcattggctcacgcttgtaatcccagcactttgggaggccgaagtgggcgaatcacctgaggtcaggagttcaagaccagcctggccaacatggtgaaaccccatctctactaaaaatacaaaaattagccgggtgtagttgtgcatacctgtaattccagctactcgggaggctgaggcagaagaattgcttgaacctgggaggcggaggttgcaatgagcagaggttgcaccactgcactccagcctgggcaacagagtgagactccatctcaaaaaaaaaaaaaaaaaaaatttttgcagagcacttacagtatctgacccgagtatcccagaaacttgcttacccttttttccattgtgttagggctgctaagggttaactgagaagaatctggttcttgccgagccttgagtgtggtggagttaggacttgagaaagccttcaagtcaaaggaaatggcaggaacagttaaagaagagaaattacagggcctaggggaaaggtacaggggctgtggttggggttgagggtcttcaggaagtagtagagaacggtgaatttagccaggcaagatgatggagggccttgattgggtgggaaatttgaactcttggataggcaacagggagccattgaaggttttagacaggagttacatgatgaattctatgatgacggtggtggtggttgtgattactgtttactgaaaccctgttgtgccaggcatgagcctgaacgcttttctttcattcttactgaatcctcaccatagctcttaaaagtatgtactgtaaacaggaaaactaaggctcctagaagttaagattctccagctagtagaattccagctctggtctttctggctgggagccagttgaagagaaagtctgggtctgtgtcccgctgaccctgtatgcaaattcagcctattcatggcttgtaacaaatttactaagttgcaaccaacacattttgttgtttctttcttttgataactttggtaccaagctcctggtcctttttagaatgagataagggtgaaatgaaacaaagtcgcagcacgcattgcatcttgcaagaccttactgatgtgctgtaaggctaaatgttgattggttaaacttttgtttctgttataaacgcgtgtgtgtctatttatagaaacatttggagtcctggactgtcatggtcaagactttgtaaaccactgcacactaggcctcaataccctcttaagagatggtttgcatttgaggtggggccggcaggctcagacagctccgggtgaccctgcatggttgcggcctagagggcctcatgttccaggctgcagccacccaccctcctaaaagggtggacaagaaaagaactaggccaggggaaggaaggctgtgggacctgaggtcaggcccagcatgggagggctgcctcagagggcctggacttgcaggcgccaggtgcccagggtctccatggagcccagcaggagccaggagagatagctggcaatcaggcgaggtgggctgacggctggtatggtccgtcagggccagccaggccttgcccttcgcagaattatgtaaccctcttcttggcccttgctaaggtttggcatctctagccaaaaccctattggggggatccatggaccagtgtgaggtggagctggtggtcttttgcctttcctatcccttatagggcttgcctcgccaggttcaggcggccagtggctggcggggggctgtttattttgggctgacgggctatatttaccttgtgaagctgcgtgcgatctgatctgatccaatcttgctggggctcagctggggctctggcagtcagctgccagatctgtagggcccctggccctccccctgccacccaaggggagatttccagagcaggaagtatcctggaaaaagtggtggcatttattcctgtgactttttcatcaagctccatgacacatgccaaccgttcctttcttctcacttttttttttcccagttgcaagattttattaaaattttttttgtttgtttcttagaactttatttatttatttatttatttatttatttatttattgatcattcttgggtgtttctcgcagagggggatttggcagggtcatgggacaatagtggagggaaggtcagcagataaacaagtgaacaaaggtctctggttttcctaggcagaggaccctgcggccttccgcagtgtttgtgtccctgggtacttgagattagggagtggtgatgactcttaacgagcatgctgccttcaagcatctgtttaacaaagcacatcttgcaccgcccttaatccgtttaaccctgagtggacacagcacatgtttcagagagcacagggttgggggtaaggtcacagatcaacaggatcccaaggcagaagaatttttcttagtacagaacaaaatgaaaagtctcccacgtctacctctttctacacagacacggcaatcatccgatttctcaatcttttccccacctttcccgcctttctattccacaaaaccgccattgtcatcatggcccgttctcaatgagctgttgggtacacctcccagacggggtggtggccgggcagaggggctcctcacttcccagtaggggcggccaggcagaggcgccccgcacctcccggacggggcggctggccgggcagaggctgcaatctcggcactttgggaggccaaggcaggcggctgggaggtggatgttgtagcgagccgagatcacgccactgcactccagcctgggcaccattgagcactgagtgaaggagactccgtctgcaatcccggcacctcgggaggccgaggctggcggatcactcgcggttaggagctggagaccagcccggccaacacagcgaaaccccgtctccacccaaaagatacgaaaaccagtcaggcgtggcggcgcacgcctgcaatcgcaggcactcggcaggctgaggcaggagaatcaggcagggaggctgcagtgagctgagatggcagcagtacagtccagcttcggctcggcatcagagggagactgtggaaagagagggagagggagactgtggggagagggagggggagggagagggagagggagagggcctttcttctcactttgaatcattcttttacacggccccctgaaccctgtggcattgaacttggttccaagtgtgggcacatacagcctgcctaagtggacggagttccaggaatcgtagggtctggtgagggtaatgggctttcttccggagcagggcagcaggccctttcctcttcctcggagagcccagcctctcccctctccccagcccaaaacctcagggctggcgagtgtgggtgtccccgggccgtgtgcgtcccaacagctgcactgctttctggccctggagcctgtgggtgtgacaaatggtgtggaatgtggtggtctctgtcctgtggaggtggccatcaggccaggtgtggcgtggggtacacatccttgtggccgaggtttggagctggagctgcctctaaatatagcaagcgaggccccgagtcggggttggaaggtgggtatgtggggtccctgtctccagtctgagctgagggtatccaggcactatttctcttccatccacttcaagctctgtttttctccttttttcttttcttttcttttttttagagatagagtcctgctctgttgcccaggttgtagtgcagtgatgggatcatagctcactgcagccttgaactcctgggctcaagggatcctcctgccttagcctctcaagtagctgggactggccaggtgcggtggctcacacctgtaatcccagcactttgtgaggctgaggtggggagatcatctgaggtcgggagttcaagaccagcctgagcaacaaggtgaaaccccgtctctactaaaaatacaaaagttagctgggcatagtggcgggcgcctgtaatcccagctactcaggaggctgaggcaggagaatcgcttgaacccgggagggggaggttgcagtgagccaaattgtgccagcgcactccagcctgggtgacagagtgagactccatctcaaaaaacaaaaagtagctggggctacagtcatgcccactatatccagctaatttttttttctttgtgtgtgtgtgtgtgtctgtgtgtgtgtgtgagatagggtctcactttgttgcccaggctggggtgcagtagcgcaatcacggctcattgtagcctcaacctcctgggctcaagggattctctcacctcagccttctgagtagctaggactacaggtatactcactacacccagctaattttttgattttgtagagatgaggtctcactgtgttgcccagggagatctcgaattcctggactcaagtgatcctcctacatcagctccctaaattgctggattacaggtgtgtgcctgctttatgtgtttttctttctttctttctttctttttttttttttttttttttttagacagagtttcgctcttgttgccctggctggagtgcaatggcgcgatcttggttcactgcaacctccacctcctgggttcaagcgattctcctgcctcagcctcctgagcagctgggactataggcacgcattaccacgcccagctaatttttgtatttttagtagagacggggtttcaccatattggccaggctggtctcgagctcttgacctcgtgatccacccacctcggcctcccaaagtgctgggattgcatacgtgagccaccgcgcccggccctgtgtttttctttttaaccaagatttattgagctgctgtagctggccctatgctaaacatctacatatgaaaactacatttaatctcagccaccatgtgagggaggtgctcctttgcccccttcatacccgggctgtggagccaagtggacttggttttgaatccagcagcagagtaaccctttggacagctgacatcacctttctgagcctcagtttcctcgtttgtaaaagaagaataatggtattagtaactcgtgggattctcttttttttttttttttttgagacagaccatgctctgtcaccaagactggagtgtagtggcactatcttggctcactgcaacctctgcctcccgggttcaagcgattctcctgcctcagcctcccaattagctgggattacaggtgcccaccaccacaccccagctaattttttttttttttttgagatggagtctcactctgttgcccacaccggagtatagtggcatgatttcggttcactgcaacctccatgtccaggttcaagcgattctcatggctcagcctcccaattttctgggattacaggtgtgtgccaccacactcagctaatttttttgtattttaagtagaggcagggtgtcaccatgttggcccggctgatcttgaactcctaacctcaagttatctgcctgcttcggcctcccaaagtgctgggattacaggcatgagccaccacacctggcccacccctgactaatttttgtatttttagtagagacagggtttcaccatgttggccaggctggtcttgaactcctggcctcaagtgatccaccctcctcagcttcccagtgtgctgggattacaggcatgagccactgcccctgtcagcctctattttataggtgtggaagctgtggcacagagaggttgagatccttatccaaggtcaacagctttgtaagtggaggagccagtttgcagccagctgtgtggtttgttctgtggtcctgactgcatagagggggagagacactacctcgggggtggctggggcacaggcgaggcaccaagtagggagcagaatgggactgcagacctgtatacaggccccccatacacaccccagcggccagctcagggcctgagccacctaaaggctccagacgtttccagtgagtggatcagtagcagtggacctggggcagctcagcaacagccgctgctgcttgtcctgtcttgggtttcaggttccggggtggcaggccagagcaggaagaggaagtgctgggggtggggaggtggcaggagacactggctgctggagggggctcccctcttccaggtgtaggcaggcctgggggcagagacaagggagctgcaggatgtggccctccatctgccgtgccctggtgacctaccccacgtcttgtgtccagcacccacatgtgctgagataccctcacccgcatggactcagaattctcagggctggctttcggccaccaaagatcttaaacacggtagaaatcctgatggcattgccccaaatttatatgagcttgaaatgttttgtaaacgcctggactgagaagtacatactgccatccactgtgggcattcttagacaaaagcctgtgaatcctgttgaaattgtcagaaaagtcctgtgaataaatgatacttggctgttttaaggagagtgtccaccatcttcagattctcagagatgtccgaccccaagtcagatccagattgtgaccttcatctcgctgcagcccctttttttatgggtgttgaggagggtcttggccaaggttaccggtaaacaggcagttaaggagctggaacatgggtttgccagtttggcctggcgttctttatgcctgagtgtgcccctctggaggatgttgtaaagagactgtggggtggggatgaggttcctaaactgagtaataatgaagctcacttttattgagggtttgttgtgtgccggacactacctgttttacatggattgactctcttaatcctcagaagaagccttcgaggcttgtcccattattcccattttacagatgtgaaaacagaggcacagaaagttgaaagcttgccagagtcatacagcccaatcaagggcaggggagtcagggttcaagccaggcagactgtaaaaggagagcggtggggaaggaaggagtccacacccctacctagggcaggaggggtgaggacaggaattccctgcctgctgggggccaggctagtgtgcggctaatggagaaacttcaacattaggcttgccgctctgagcctgccacatatatagcaaacaccttataaagagtcactgtccttgatggaatgactgccataggtatccgccatctccctacccctgtccctccagccctcatgggttgaccatggttgtgtgcccactgcccgctgctcctggcccagatctgataagctggctcctttgcatgggggtgacttgcaaggcccaggtgaggagcagcgggtgtctggcccggagcatgcccctggtgacctcgggtccacaggtctccaggaactgggggcagctggtgtgtcagccctggggctggggaggagacagccccagggtgtactggccacccagcctgcaccagggctgggcccgtcgcttctgggaagcctgctctgtatgtcaggtcgtgcgctgtgttctcagaagggcctgagtgtgccctgggagggcagccaccacccctctgcatgtccccagagcagggcatgaaggagaagcctctgcttgggtgtgaggatgtggggccagcaggcatgatggctggcgattgctttttttaatgcttttattttaaccatagtaaaatacgcataacaaaatttaccattttgaccactgatggtggtggttggtactaattttaattttattattattattattattattattattattattattattattattgagttagagtctcactctattgcccaggctggagtgcagtggcacgatctcagctcactgcaacctccgccacccgggttcaagtgattctcctgcctcagcctcccaagtagctgggattacaggcacgtgccaccacgcccagctaatttttgtattttcagtagagatggggtttcaccatgttggccaggctggtctccaactcctgacctcaggtgatccacccgcctcaatttcccaaagtgctgggattacaggcatgagccactgggcccggcctgattttattttttttagagacaaggtcttgctctggtgcccagactggagggccatggggcagtcatagctcaccaccacctcaaactcctgggctccagcgatccttccccaccagactcccaagtagctagaactacaggtgtgcaccaccaccccccagctaattaaacaaattttttttatagaggtggggtttctctgtgttgcctggctgctctcgaactcctggcctcaagcagtcctcccaccgcagcctccacaagtattgagattacaggcgtgagccactgtgcctgtcctgcggctggtttctaatacctggctaggtggtaatagcagtgttttgggaggtggttaccatgtgccagctctcatttaattgttaaaataattctatgaattggctgggtgtggtggctcacacatgtaatcccaacactttgggaggctgaggcaggcaggtgggtcacctgaggttgggagttcgagaccagcctggccgacatggtgaaaccccgtctctaccaaaagtacaaaaaagtagcaaggtgtggtggcgcatgcctgtaatcccaggtactcgggaggctgaggcaggaggatcacttgaacccgggagtggaggttgcagtcagccgagatcgcatcactgggctccagcctgggcaaaagagcacgactctgtttggagaaaaaaaaaaaaaaaatcctatgaactactattatatccctattttacggataagaaagtcgaggctcaggccgggcgcggtggctcacacctgtaatcccagcactttgggaggctgaggcgggcggatcacgaggtcagaagatcgagaccatcctggctaacacggtgaaaccccgtctgctaaaaatacaaaaaattagccgggcgtggtggcaggcgcctgtagtcccggctacttgggaggctgaggcaggagaatggcgtgaacccgggaggtggagcttgcagtgagccgagatcgcgacactgcactccagcctagcttgggcaacagagtgagactccctctcaaaaaaaaagaaaagaaaagaaaatcgaggctcagagaggttaagtgactttcccagggttgcacagttagtgagtgccagggctgggattcagatccaggcagccttggctcttgctttctgtagggctttctgccactctcatccacaagtggataggccttactatccccattgtagacatatgaaaactgaggctcggagaggccaagcgactggccagggtcccagagcctgacaggaggagagctaggactgaagagtagtagtgtgggctgggaccgctggcactcatcctgcctgtccccccgcaggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcagccaggccagccaaggcccaccagcaagccccccgccagtggcgcagcagccaacgtcagcaccagcggcatcaccccgggccaggcagctgccatcgcctcgaccaccatcatggtgcccttcggcctgatctttatcgtcttcgccgtccacttctaccgctcactggttagccataagactgaccgacagttccaggagctcaacgagctggcggagtttgcccgcttacaggaccagctggaccacagaggggaccaccccctgacgcccggcagccactatgcctaggcccatgtggtctgggcccttccagtgctttggccttacgcccttccccttgaccttgtcctgccccagcctcacggacagcctgcgcagggggctgggcttcagcaaggggcagagcatggagggaagaggatttttataagagaaatttctgcactttgaaactgtcctctaagagaataagcatttcctgttcttccagctccaggtccacctcctgttgggaggcggtggggggccaaagtggggccacacactcgctgtgtcccctctcctcccctgtgccagtgccacctgggtgcctcctcctgtcctgtccgtctcaacctccctcccgtccagcattgagtgtgtacatgtgtgtgtgacacataaatatactcataaggacacctccttcccgtgtcttgtatttgttgggcctgggctactgctcaccctggttaggtgagcctctaggaaaacttaaaacaaattttaagccaggtatggtggcacatacctgtggtctcagctattcaggaggccaaggcaggaggatctcttgagcccaggagtttgagaccccatctcaaacaaaaaatacaaaaattagccagccacggcgcctgcacttccagctcctttgagagactgaggcaggaagattgcctaagcccaggaggccaagtctgcagtgagctatggtaacaccactgcactccaacctgggcaacagagggagactctgtctctaaaaaaatagaaaaatttgccctgcatggtggctcacgcctgtaatcctagccctttggaaggccaaggcgggcagatcacttgaggtcgggagttcgagaccagcctgaccaacatggagaaaccccatctgtactaaaaatacaaaattagctgggtttggtggcgcatgcttgtaatcccagctactcgggaggctgaggcaggagaatcgcttgaacccaggaggcggaggttgcagtgagctgagatcgcgccattgcactccagcctgggcaacaacagtgaaactccgtctcaaaaaaaaaagaaaaaaaaattcaacattttattttgagaaatttcaaacctacaaaaagttgcaggaatagtgtctatctgaaatacatattcagtcttttctttagaagtcttttttttttttttttttttaaatagagcctcactctgtcacccaggctggagtgcagtggcgcgatctgtaaaatcactgtgagcactgacttagcacatactggaccgttgctcctaggagaaatacagggttgcgttcctgtgagctttggtcgtgatattttcatcagctgatcaatatgtaatcttgttttatgtgtatttctgtttaaagattcatatatatatgtgtatatatatatatggttgagttgttaccgttgaactcacagcccacaggactagaacacatgcctaaataaagtttatctaacatacttgttttctccatatgtatatccatcataaccttcttgcactgaggaatattagataatatatatatgtatttatgtattatacatatatatatatatttttttttttgagacagagttttgctcttgttgcccaggctggagtgcaagggcacgatctctgctcactgcaacctccaccttctggtttctttcttttgtttgaaacaaagttttaccttgttgcccaggctccagtgcagcggcacaatctcagcttactacagcctttgcctcccaggttcaagtgattctcctgcctcagcctcttgagtagctaggattacaggcatgcaccaccatgcccagctgatttttgtgtttttagtagagacggggttttaccacgttggccaggctggtctcgaactcctgacctcaagctatccacccgcctgggcctcccaaagtgctgggattacaggcatgagccactgcgcccagcccaccttctggtttccagcgattctcctgcctcagcctcctgaatacttgggattacaggcaccggctaccacacccggctaatttttttgtatttttagtagaggcaggggtttcgctgtgtgggccaggctggtctggaactcgaccccgttatccacccaccttggcctcccaaagttctgggattacaggcgtgagccactggacccggcctattattatatatatatattttgaaacagtctcactccatcccccaggctggagtgcagtggcacaatctctgatcacatgcagcttccgcttcctaggttcaagtgattcttctgcctcagcctcccaagtagctagaatcacaggagcgcgccaccacacccagcaaatttttgtatttttagtagacgggatttcaccatgttggccgggctggtctcgaactcctgacctcacgttatctgcccgcctcagcctcccaaagtgctgggattacagacatgagccactgctccctgcctattttattttagagacagggtttccctctgtcacccaggctggagtacagtggcatgaccatagctcactgtaaccaccaactcctgagctgaagtgatcctccctgcagtgagctctggtcactccacctcccaagtagctaggaccacaggcacatatcaccatgcccagctttttttttttttttttttttgagacggagtttcactcttgttgtccaggctggagtgcaatggtgcgatctcggctcacagcaatctccacctcccgggttcaagccattctcctgcctcagcctcctgagtagctgggattacaggcatacgccaccacgcctagctaattttgtatttttagtagagatggggtttctccatgttggtcaggctggtctcgaactccagacctcaggtgatccacccacctcggcctcccaaagtgctgggattataggtgtgagccaccgcgcccagctttctttttttttttttgagatagaggtttactcttgttgcctaggctagagtgcaatggtgcgatctcggctcactgcaaagctggcaaagtcacagacactgactgaagactgaggagggactgtgtctaccttccccctgaggcatgtgctgtagttaccggtatggtccttcatgcctaaatacttcctcctaaaactaaggacatttccctacatgacgacaatccagtgatcaaatccaggcaattatcagatagaacattcctgttcagtttcccaaatgtcccagtaatgtcccttctagtgacgttttttccacttgaggattgtgtgtcacaacgagatatcacttcctagtctcctttattatttttattttattattatttttttgagacggagtctcactctgtcgcccaggctggagtgcagtggcatgatcttggctcactgcaaaccccgcctcctaggttcaagcgattcttctgcatcagctgcccgagtagctgggattacaggtgcccaccaccacgccgggctaatttttgtatttttagtagagaaggtgtttcaccatgttggctaggctggtctcaaactcccagtctcaagtgatcccctcgccttggcctttcaaagtgttaggattacaggcgtgagccaccgtgcccagccttagtctcctttaatttggaacaattcctcagccttttttttgtttgtttgtgtttgagacagggacttcccatgtcacccaggctagagggcagcctcaacctcctaggctcaagcaatccttccacctcagcctcctgagtagctgggaatacaggcacatgccactacgcctggataatgtttgtattttttatagaaacagggcctctcttgcccaggctggtcttgaactcctggcttcaagggatcctcccaccttggcctcccgaactgctgagattgcagctgtgagccacagcacccaggctagcctttgtctttaaaaaactttttggctgggctcagtggctcacgcctgtaatcccagaactttgggaggccgaggcgggcggatcacgaggtcaggagatcaagaccatcctggctaacagggtgaaaccccgtctctactaaaaaatacaaaaagttagctgggcgtggtggtggctgcctgtagtcccagctgctcgggaggctgaggcaggagaatggcgtgaacccgggaggtggagcttgcagtgagccgagattgcaccactgcactccagcctgggcgacagagagagagtccgtctcaaaaaaaaaaaagaatacaaaaattagctggtcgtggtggtgcgtgcgtgtaatcccagctactcgggagactgagacaggagaatctcttgaacctgggagctggaggttgcagtgagctgagagcgtgccactgcactccagcctgctggacagagcaagactccaaaaaagaaaaatcattatgtgcagctgctattctacgcactatgcatcaactcatttaaccctcagcacccttatatggtgggtacctttttttttttttttttttttgagacggagtctctcacttttgtcacccaggctggagtgcaatggcgcgatctcggctcactgcaagctccacctcccaggttcccgccattctcctccctcaacctcccgactagctgagactacaggcgcccgtcaccatgcccggctaattttttgtatttttagtagagacggggtttcaccacgttagccaggatggtctcgatctcctgacctcgtgatccgcccatctcggcctcccaaagtgctgggattacaggcgtgagccaccgcgcccggccagtggtgagtactattattatctccattttgcagaaggtaaaattcatgcccgtggaagttaagtaaattgccaggatttgaacctggatagtctggctcagagcctaggccctcacaaagatcttacctgggctgattctggctttgctttaaatggagggccgggtgtggtggctcatacctgtaatcccagcactatggaaggctgaggcagctgtatcacctgaagtcagcagtgtttgagaccagcttgaccaacagggtgaaaccccatctctactaaaaatacaaaaattagccaggcgtagtggagcacacctgtaatcccagctacttgggaggctgaggcaggagaatcatttgaacctgggaggcagaggttgcaatgagctgagatcacggcattgcactccagcctgggcgacaagagcaaaactcggtcccaaataaatatatacatacatacatacatacatacatacacacacacacacacatacatacattaaagaactgttggaagccctagctgcatggggccaccttcttacagagcaacaatgagcagcagctgcactgcagccacactctccccagggcctattttttccacagtccccagcactccctcttgtctcacaccaggccgccttcactcatgtgactttcttggcctcattaggcctttgagtttgagatttctgtctgtcccaaacatctaatatagaaagtggttatgctcacgcctgtaatcccagcactttaggaggatgaggcaggtagatgacttgaacccaggagtttgagaccagcctgggcaacatggcgaaaccctgtctctacacaaaatacaaaaattagttgggcatggtggcacgcgcctgtaatcccagctacttgggaaactgaggctggaagatcgcttgagtctgggaagtagagggtgcagtgatccttgattgtgctactggactccagcctgggcaacagaccctgtctcaaaaaagaaaaatttaaaccaagtttttaattagttttataaaagaatgtagtctggctgaagtgaaatgtgggtggagcatcagcaggcctgatatttctctttctcctatcattgttgtcccaggtgcttcacagaaccccaagactctggagaaacacaatttggaaagtgtggccttaagttcttgtcctgtgcggactgggacacagtgaacggcagcttcatagtctgggtggggaaggggaccctccttttccctccagcagcatttgcagtcctgtgtcacatgctccaggatccacacttcttttttgtttgttttgttttgttttgagacagagtctcattctgtcgcccagactggagtacagtggtgaaatcttggctcactgcaacctccgcctcccgggttcaaacgatcctcctgcctcagcctcccaagtagctgggattataggtgcacactaccacgcctggctaatttttgtattttttggtacagatggaggtttcaccatgtcggccaggctagtctggaactcctggcctcatgtgatccacccgcttcagcctcccaaagtgttgggactacaggcgtgagccaccacgcctggcctaatccacacttttgaagggtatcaagtagatgagggagggacctctatgaacccccactgctccccagagaagtcctggacttttttttttttttttttttttgagaccaagtctcactctgttgcccaagctagagtgcagtggcgcgatttcagctcactgcaacctctgcctctcgggttcaagggattctcctgcctcggcctcccgagtagctgggactacaggcatgagccaccacgcccggctaatttttgtatttttagcagagatgggctttcaccacactggccaggctggtttccaactcctgacctcaggtgatccacctgcctcagcctcccaaagtgctgggattacaggcgtgagctaccgcacccggccgtcctggacatcttttacgtattcccctaggacagccacctccctacttctgtagggatgcaagatccagctggaattatttctgggctaggatcctgttgtcttgggttcttgttcctaaactggccgggactcgactcatcagccaggcttgtcaaatgcaggtttcgaagcccttggaggattctgatttaagtaggtctgggtgtgtctttggaatctatttctaacaagcaccccaggtgctctcacacccaggcaacttagacaaacactcctagttaggagcacagtctctttaacttcatttttatttacattttgaatacataatacattcacatggttccaaacacaacactgaaaagttgttcttcagctcctgttcccaagtccccaattctccccagagacaaccatctttgccagtttattgtgtttaaatctagcgacattttgtacataaacaagcacaccctcttccaacattttttttttttttgagatggagtctcgctctgttgcccaggctggagagcagcggcacgatctcggctcggcgtgatcttggctcaccactacctccgcctcctgggttcaagtgattctcctgcctcagcctcctgagtagctgggattatagatgcactccaccacacccagctaatttttgtatttttagtagagacagggtttcaccatgttggtcaggctggtctcgaactcctgacctcttgatccgcctgcctcggcctctcaaagtgctaggattacaggcatgagccaccatgccctgcccccactttttctttaaaaatggtaaccctttacactcttctgcacattaagaacagctagacctaagtttaagcacagtactagcttgatgacttgtgatcctccctgaacctgtttcctcctctaaaatggcagtaatagcaagacacctcactgttgtcggggagtgccaggagagaatgcttgctgcgaagctgtcagcacacggcctggaatagagtgagtgctccatatctggaagttatgattaattctgcatcctagggtggccaggcggggcttcccagtgccagaggtacagactgagaaccacacccttggtgaggccaccctacttatgacttagggggcagagagaacctcagtaaggtcagtggtgtttattgaggctttactatgtgctatgtgcctcttgctgaggtcttagctagaatcgtggtatttcagggtttctcaacattttttttttttttttttttggagatggtgtctcgctctgtcgcccaggctggagtgcaatggcgcaatctcggctcactgcaacctctgccacccaggttcaagcaattctcctgcctcagcctcccaagtagttgggattacaggtgctgaattcttttttttttttttttttgtagcatggtgatattgcactatgcttaaccgcatccctggtctctactcactcaatgccagtagccttcttcctgcctgttgtgacatcaaaaatgtcatacattgccaaatgtccccaagaaagagtggggctgagggaggacaaaatcctacccacagtttaaaaccaccgccttatctcatcctcacagtaactaaccacagtaaccccaggtggtaagtactgttaatgccccatattgcaagtgaggagaccaacagaacagagtttgggtgacattcctgagaacacacagctccgagattcacgcccaggcactgtgactccagagctcacctgcccattatgcaagaacatctgatcttggctgggagcagtggctcacacctgtaatcccagcactttgggagtccaaggcgggcagatcacgaggtcaggagatcgagaccgtcctggccaacatggtgaaaccctgtctctactaaaaatacaaaaaattagccttggtggcaggtgcctgtaatcccagctacttgggaggctgaggcaggataatcgcttgaaccagggaggcgaggttgcagtgagccgagattgtgccactgcactccagcctgggtgacagagcaagactctgtctcaaaaaaaaaaaaaaaaaaaaaaaaaaggccaggctcgatggctcacacctgtaatcccagcactttaggagactgagcggggcagatcacgaggtcaggagttcaagaccagcctgaccaacatggtgataccccatctctactaaaaatacaaaaattagctgggctcagtggcatgcgcctgtaatcccagctactcaggaggctgaggtaggagaatcgcttgaacatgggaggcagaggttgcagtgagctgagatcgcgccactgcattccagcctgggcaatagagggagactacgtcttaaaaaaaaaaaatcacctggggaattttctgaaagctcaggcctcaccccagatgaattagatgtgaacctgtgaatctctgggcatgggacctgggcttccacagctttgcaaagtttccatgggatttcaaggttgagagccctgtgttaggctgagaagtggtcaggaaattttatgatctccaatcttggcagccagtgtggcaggatacaggccttctctctgggtcatctgggttatctcacaggggacctgccatttctttctctttttcttttcctttcttttatattttaaaatttttttttgtagagtcagggtcttgctatgttgcctaagctggtctcgaactcctggcctcaggcagtcctcttaccttggcctcccaagttctgggattacaggaatgagccactgcacccggcctaagacctgcccttactcccccaagctaaacccaggcagcccagccaaggcaggtcttccctcttctaaacctgaaagccctgctgagccccctcgagtgccccatccttatcccataggcccaggcttataccacttggttattctttattctcatgcattccaggatgagatcagagaatgaagagggtcccgaggacagcctgggagagaagcaccagccttcctattggcaagaaatgacccagtgctgggggtagtgaggggccctatgagggtgccggggtcatgcctggagctgctgctgactcctccagccagagccgcactggtctgagcgatcgggtgacacaggcttgaatttctcctgaagcgtttgtgttctctgcaggggagagagggatggtggagctggcatttgaaagacgctgttaccaatgtcctctcctcaaaccctcacatcatgcccaggaggacttggactggacaagaccccactggacagatgtgtaaatggaggctgaaggccaactgccttactcaagggtgttcagaagccaggagaccacctcctttcaaccccgtgcccagctcagttgtgtggcccagggtgcaaatagaaggtttcaggcaggactggaatgcactggtcttcagtgaaatggtttgtttgtttgtttatttgtttgtttgtttgttttttgagacacagtctcacccagactggagtgcagtggcacagtctcagctcactgcaacctctgcctcccaggttcaagcaattctctgcctcagcctcctgagtagctggaattacaggtgccctcccccacgcccagctaatttttgtgtttttagtacagatggggtttcaccatcttggccaagctagtcttgaactcctgacctcgtgatccacccacctcggcctcccaaagtgctgggattacaggattataggattactgtgcctggcgagttttgttttttttgagacagagtcccattctgtcgcccaggctggagtgcagtggtgcaatctcagctcactgcaaccttcacctcccgggttcaagtgattcccctgcctcagcctctcgagtagctgggactacaggtgcctgccaccacacctggctaacttttatatttttagtagagatggggtttcaccatgttggccaggctggtctcaaattcctgacctcaacttatccgcctgccttggcctcccaaagtgctgggattaaaggcaagagccaccacagccggtcaaaaggggttttgaagaggaagcgtcaaaaaggacacctgggactgtaggaggcaagaagcctgccctcgatggtcttgctcccccactccccgaccccaagccctttggagaccccaggagatctgtgcattgttctgggccctggagcagcctcagccacatcagaacttggcaggtgtgcccacccctcacctggcatcaatctccttcctctgtcttcctgaacatggccaaggcctccgccagcacaccatcagggtctaggattttgacctggagggaaataccaagaagttggtttggagagcatggaggacccaggctgccagccttgactggcgctgcctgccgtgtggggtgctgccagcctcatcaggagccacctcccttgctagggatgccctgactgaaaacaaaaccttgtctgttccctgtctgggcttggctgggcctggcagatctggcctgggcatggctggggatgctggcttctgtctctgtctgggtgtggccacagggccctggtgagggctgcccacctcaggaatggggaactgagtgggctcaggggcctcgtcacggccaaagtcgatcatcgtcccgcatttggccatattgtccacccagaagccttcaaacagctggccgtggtccagatggaagaaacgccccgccccgttcttcatgcctctctcccagcagccctcgtagcggttcccgttctctgggggaaaggacagggaggtgtggtgcagggtacaccaaactaagctagacccccaggggtcgctcttccagagctcagtgcaaatcctaacacattgggaggccaagatgggaggctcataagcccaggagatcaagaccagcctgagcaacatagtgagaccacatctctgcaaaaaaaaaaatttttttaattagctgggcatagtggtgtgcacctatagtcccagctactctggaggctgaggtgggaggatggcttgagctcaagaagttgaggctgcagtgagctgagattgccccacagcactccagcctgagtgacaacctccgcctcccgagttcaagtgattctcctgcctcagcctcctgagtagctgggattacaggtgtataccaccacatgcagctactttttgtatttttagtagagacagggtttctccacgttggccaggctgatctcaaactcctgacctcatgatccgcccgcctctgcctcccaaagtgctgggattacaggcatgagccactgcgcctggctttttttttaaaaattgagatataagttcatataacataagttggccattttgaagtgtatagttcagtggcatttagtacattcacaatgctgtgcaaccattacctctatctagtttcaaaacgttttaatcacccgaaaaggaaaccctgtgcccattaagcagccattccccacacccactaatgtcaccacccccatagtcctaggcaaccattagtctactttctgtctctgtggaggtgcgtgtcctaaaaatttcatataaatagcatcatatatatgtgaccttttgtgactgaggggagagatagggtgctcttcctctcttgtcccacattgacacaagggtcagtgtgggacaagagtgtggggtgtggcctgtctctgtgggaccaaatgggcgtggccagctcgctgccgggggcgtggtcgggtgggcggggccggcgggggtggggcactcactcaggcgcagcatgccctccccgttgggcttgtcgttctcccactgtccctcgtagatgtcgccgttgctgtaatacatgcggccccacccgctgcgctggctgccacaccagtcaccctcataatactccttgggtccgaaaaactggatcccataaccctgaaagtacgaagatgctactactcagggcgccccccaacaccaccaggaaagcccaccgtcttcccaggcacccctagagccacacaccccagcttcttccttcttcctcagccctacgtccaagcggagggtcgtgtcttagcggagtatcctgtcttttttgtttgtttgtttgtttttagacagagatccactctgtcacccaggctggagtgcagtgatgtgatctcggctcactgtaacctccgtcccccgggttcaagtgattctcctgcctcagcctcccgagaagctgggattacaaacacgcatcaccacgccctgctaatttttgtatttttaggagagacagggtttcaccatgttggccaggctggtcttgaactcctgacttcaagtgatccgcccgcctcagcctcccaaagtgctgggattacaggtgtgagccatagtgcccaccgataatgtcttgaaggatctccccatgctctgtttctctctgtccctctgctggcgcctccatgcagccagtcattaaactccctccttactgacccaccctctcctctctcatccccaccacaccatgcgccatgcagcccctacagcgccggaggactctttaaatcgcccatctaggctgggcgctgtggctcaccggctcactttaggaggctgaggtgggtggatcacttgaggtcaggagtttgagaccagcctggccaacatggtgaaaccccatctctaccaaaaatacaaaaaattagctgggcgtgatggcggcgcctgtgatcccagttactggggaggctgaggcaggagaatcacttgaacctgggagatggaggttgcagtgagctgagaatgcacggtagcctaggtgacagggtgagacgctatctcaaaaaaaaaaaaaatcgcccatctgatcatgctcgcccccctcccactcttattttgacccagaaaaatgaacataaacaccatacttagcctggcgtggtggcttacgcctataatcccagcactttgggaggccgaggcagatggatcacctgaggtcaggagttttagaccagcctgaccaacgtggcaaaaccctgtctctattaaaaatataaaaaattagctgagtgtggtggtgggcacctgtaatactagctacttgggaggctgaggcagaagaattgcttgaacccaggagtcggaggttgcagtgagcccagatcgtgccattgcactcccacctgggcaacagagcaagactgtctcaaaaaacaaacaaacaaacaaacaaaaaacaccacacttaggctgggcgctgtggctcacgcttgtaatcccagcactttgggaggccaaggcaggcggatcacaaggtcaggagatcgagaccatcctggctaacatggtgaaaacccgtctctactaaaaatacaaaaaaattagctgggtgtggtggtgggtgcccgtagtcccagctactcgggaggctgaggcaggagaaggccgtgaaccaggaggcggagcttgcagtgagccaagatcgcgccactgtactccagcctgggcaacagagaaagactccgtctcaaaaaacaaaaaacaccacacttagtgtccaaccacagggaaacaaggtctgtttgcagatcacacccgaggaggttgggttgagaagccaagtctactcctcaaaatcctgatttcccacatatccctgagggcccccgcaactcttctcctcactcctgtcccagggcagacacactctgctgttttacatctcgttccctttgctatttttttgcctcccatctttctcccttttttttcttcctggctaactcctagcatcttttagtgtcacctcctacaggaagcctgccctgaattccctctttttgagacaagtcttgctctgttgtccaggctggagtgtagtggcaccaccatagctcactataacctcagcctcctgagatcaagcaatcctcctgcctcagccttttgaaaagttgggactacagatgtgcaccacaacatctagctaattattattattatatagttttatttatttatttatttattttgagatggagtctcactctgttgcccaggctgaagtgcagtggcgtgatctcggttcactgccacctctgcctcctggcttcaagcgattctaatgtctccgtctccctagctgggactacaggtacacaccactatgcccggctaattttttttgtgttttttatttagtagagacgggggtttcaccatgttggccaggctagtctcgaactcctgacctcaagtgatacacccctgtctgcctcccaaattgctgggattacaggcgtgagccattgtgcttagccttctgtcctgaattctctaggctgggcttgatgaccttcctctgggcaccctgtgaaaacatccctcctaataccatattctacgtaaattggttattcagggctttctcttcctacttgccaataaacaatttgggagcagagaacatgaaaccctgggctctaccacttgctagctgtgtgactccaggcaagactgaaactgcctttgcaaaattatgactgagacagtgaaagagatttaatttaactgactccatcttgcttctaacctctaagctgtccatgtccattcctgggcttaagctgaactaactttgggagaaacttatagtttatagtttaaacaaagacagtaacagccctttcccgaagacctccttcttgcctggggactagattgcctttgtaggattaacattagctacaagattataaattatggtttaggagtcatgcagctggaagctacaagattctgatcctcactaaactgctcctaagatcaatgcttaagatattttgcagaccttgcacttgatggatcagctggcactacccaaatcaataaactgatttatgtgatcttgtggctccccacctaggaactgactcagggcaagaagacagctttgactccctgtgattttatctctgaccaatcagcacgcctggcttactctttcccccacccaccaagttatccttaaaaactctgcagccagaatgttccgggagactgatttgagtaataatacaacccagggccaggcgcggtggctcatgcccgtaatcttagcactttgggaggcccaggctggcggatcacctgaggtcgggagttcgagaccagcctggccaatatggagaaactctgtctctactaaaaatacaaaattagcagtgtgtggtaccacatgcctgtaatcccagctactcgggagactgaggcaggagaatcgcttgaacccaggaggcggaggttgcggtgagccgagatcacaccattgcactccagcgtggcgacaagagcgaaactccatctcaaaaaataaataaattaataataataataataaaactcaggtctcccacacagccagttctgtgttaattactctttctctattgcaattcccgtcttgatgaatcggctctgtctggggagcaagaagacagctttgactccctatgattttatctctgaccaatcagcactcctggttcactgtctttcccccacccaccaagttatccttaaaaactctgctgctggaatgttcggggagactgatttgagtaataatacaactcaaggccaggcaccggcaaggtgaaccccttgggcagttacaagacacctcatctcttcgagcttgtttgcttatcaataaaatgggcatgaagatgatgatgatgatgagctgttgctgtgagagttaactgaatattaaatgtgtttcaggccgggcgcggtggctcacgcctgtaatcccagcactttggaaggccgaggcgggcggatcacgaggtcaggggatggagaccatcctggctaacacggtgaaaccccgtctttactgaaaatacaaaaaattagccgggcgtggtggcgggcgcctgtagtcccagcaacctgggaagctgaggcaggagaatggcgtgaacccgggaggcggagctcgcagtgagccaagatggcgccactgcactccagcctgggcggcaggatgagactccctctcaaaaaaaaaaaaaaaaaaaaaagttttaaatatgtgtttcaggctcacagtagcagttgctcagttgcaatactgtttattattagtgtctctgggactgatggtgaggattcctccacacttgtgggagtagatggagggaacctgtgcccagcttgggtgtttccgggctgagcacgcgcctctggctggcttcggccccccgggaccctcctctcctgtcccctgctctcttgctcccacttttcctacctcccttttattcaggcccagcagaaaggggattctcagaaagtgatacctggggaaaagaaagccaggaaacgagggggaaaagggtggaggcgacttcg

SEQ ID NO: 2 - DNA, 52 bp, Homo sapiens
aagagttacgtttcaccccgccaaccggcaatgtcacatgctcgtcactcat

SEQ ID NO: 3 - DNA, 52 bp, Homo sapiens
tcttggcatgagcagttccctcgccgtcctggaaagctagatcctgtctgtt

SEQ ID NO: 4 - DNA, 52 bp, Homo sapiens
caagttctagagctggttcagaagtttcctgtaatttctatgaaaatgagtt

SEQ ID NO: 5 - DNA, 52 bp, Homo sapiens
aaacgcctggactgagaagtacatactgccatccactgtgggcattcttaga

SEQ ID NO: 6 - DNA, 52 bp, Homo sapiens
gctgggcttcagcaaggggcagagcatggagggaagaggatttttataagag

(19) United States
(12) Patent Application Publication
(10) Pub. No.: US 2012/0028827 A1
(43) Pub. Date: Feb. 2, 2012

(54) METHOD FOR DETERMINING A RISK, FOR A SUBJECT, OF SUFFERING FROM ATOPIC DERMATITIS OR SEVERITY OF ATOPIC DERMATITIS FOR A SUBJECT SUFFERING FROM ATOPIC DERMATITIS AND METHOD FOR USING A SINGLE-NUCLEOTIDE POLYMORPHISM RS12313273 AS A BIOMARKER FOR DETERMINING THE DEVELOPMENT OR SEVERITY OF ATOPIC DERMATITIS

(75) Inventors: Wei-Chiao Chang, Kaohsiung City (TW); Chih-Hung Lee, Kaohsiung City (TW); Suh-Hang Juo, Kaohsiung City (TW); Wei-Chiao Chen, Kaohsiung City (TW); Hsin-Su Yu, Kaohsiung City (TW)

(73) Assignee: KAOHSIUNG MEDICAL UNIVERSITY, Kaohsiung City (TW)

(21) Appl. No.: 13/193,472

(22) Filed: Jul. 28, 2011

(30) Foreign Application Priority Data
Jul. 30, 2010 (TW) 099125309

(51) Int. Cl. C40B 30/04; C12Q 1/68

(52) U.S. Cl. 506/9; 435/6.11

(57) ABSTRACT

The invention provides a method for determining a risk, for a subject, of suffering from atopic dermatitis, including: obtaining a biosample of the subject; detecting the presence of the single-nucleotide polymorphism rs12313273 (C/T) at position 30881 of the ORAI1 gene (SEQ ID No.: 1) in the biosample; and determining the risk, for the subject, of suffering from atopic dermatitis, wherein the presence of a C allele or genotype CC of the single-nucleotide polymorphism rs12313273 (C/T) indicates that the subject is at increased risk for suffering from atopic dermatitis.

### CROSS REFERENCE TO RELATED APPLICATIONS

This Application claims priority of Taiwan Patent Application No. 099125309, filed on Jul. 30, 2010, the entirety of which is incorporated by reference herein.

### INCORPORATION BY REFERENCE OF SEQUENCE LISTING

A sequence listing submitted as a text file via EFS-Web is incorporated herein by reference. The text file containing the sequence listing is named “0911-A52307-US_Seq_Listing.txt”; its date of creation is Oct. 13, 2010; and its size is 82,236 bytes.

### BACKGROUND OF THE INVENTION

1. Field of the Invention

The present invention relates to a method for determining a risk, for a subject, of suffering from atopic dermatitis, and in particular relates to use of a single-nucleotide polymorphism (SNP) rs12313273 (C/T) as a biomarker to determine the development and severity, for a subject, of suffering from atopic dermatitis.

2. Description of the Related Art

Atopic dermatitis, a recurrent chronic inflammatory skin disease, co-influenced by genes and environmental factors, is one of the most common skin diseases for infants and children. Atopic dermatitis occurs in 3-5% of children, and has increased in recent years. Atopic dermatitis usually occurs along with allergic asthma and rhinitis. Burden for an individual with atopic dermatitis is seen in communities, families, and the physiology, psychology and economic welfare of the individual. 60% of patients fall ill during the first year after birth, and 30% of patients fall ill during the second to fifth year after birth. Most patients with atopic dermatitis usually have allergic asthma and rhinitis. Specifically, the condition for patients who have atopic dermatitis or whose family members have allergic asthma, allergic rhinitis or an allergic rash is called atopic constitution. Patients with atopic dermatitis often have higher serum IgE levels. However, some patients with atopic dermatitis may have normal serum IgE levels.

The ORAI1 gene is capable of being transcripted as a channel protein subunit of a store-operated Ca²⁺ channel. For non-activated cells, such as immune T cells, epithelium cells and mast cells, the store-operated Ca²⁺ channel is a meanest approach for Ca²⁺ entering therein. Feske et al sequenced the gene of patients with severe combined immunodeficiency syndrome (SCID) and found that the mutation of the ORAI1 gene was in relation to the development of severe combined immunodeficiency syndrome (Feske, Stefan. “ORAI1 and STIM1 deficiency in human and mice: roles of store-operated Ca²⁺ entry in the immune system and beyond”. Immunological reviews.). The research of Chang et al disclosed that the ORAI1 gene participated in the development process of the allergic response (Chang W C and Parekh A B. Close functional coupling between Ca²⁺ release-activated Ca²⁺ channels, arachidonic acid release, and leukotriene C4 secretion. J Biol Chem 2004 Jul. 16; 279(29):29994-9).

### BRIEF SUMMARY OF THE INVENTION

The invention provides a method for determining a risk, for a subject, of suffering from atopic dermatitis, comprising: obtaining a biosample of the subject; detecting the presence of the single-nucleotide polymorphism rs12313273 (C/T) at position 30881 of the ORAI1 gene (SEQ ID No.: 1) in the biosample; and determining the risk, for the subject, of suffering from atopic dermatitis, wherein the presence of a C allele or genotype CC of the single-nucleotide polymorphism rs12313273 (C/T) indicates that the subject is at increased risk for suffering from atopic dermatitis.

The invention also provides a method for determining severity of atopic dermatitis for a subject suffering from atopic dermatitis, comprising: obtaining a biosample of the subject suffering from atopic dermatitis; detecting the presence of the single-nucleotide polymorphism rs12313273 (C/T) at position 30881 of the ORAI1 gene (SEQ ID No.: 1) in the biosample; and determining the severity, for the subject, of suffering from atopic dermatitis, wherein the presence of the C allele of the single-nucleotide polymorphism rs12313273 (C/T) indicates that the subject is at risk for increased severity of atopic dermatitis.

The invention further provides a method for using a single-nucleotide polymorphism rs12313273 (SEQ ID No.: 3) as a biomarker for determining the development of atopic dermatitis.

The invention further provides a method for using a single-nucleotide polymorphism rs12313273 (SEQ ID No.: 3) as a biomarker for determining the severity of atopic dermatitis.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows the relative positions of five single-nucleotide polymorphisms in the ORAI1 gene;

FIG. 2A shows the result for inhibiting ORAI1 protein expression by siOrai1;

FIG. 2B shows the result for inhibiting EGF-mediated calcium ion channel efficiency by siOrai1; and

FIG. 2C shows the result for inhibiting EGF-mediated COX-2 gene expression by siOrai1.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

In order to realize the relationship between the single-nucleotide polymorphism (SNP) and atopic dermatitis, an effect of the single-nucleotide polymorphism of the gene for a store-operated Ca²⁺ channel on development of atopic dermatitis is investigated in the invention. In one embodiment, the ORAI1 gene (SEQ ID No.: 1, wherein the position 30881 thereof is the transcription +1 position.) is selected for investigation.

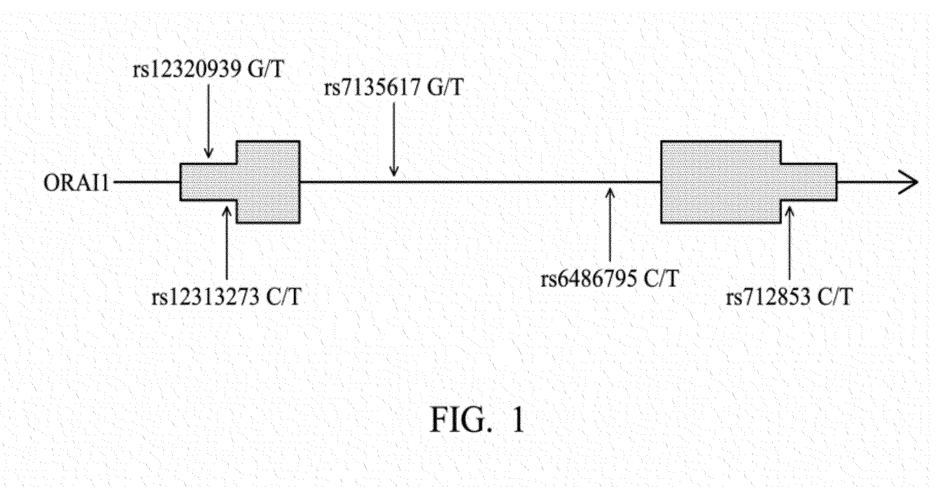

In one embodiment, five single-nucleotide polymorphisms are selected and the individual relationships between the selected single-nucleotide polymorphisms and atopic dermatitis are analyzed. The five single-nucleotide polymorphisms are rs12320939 (SEQ ID No.: 2 is at position 30567 to position 30618 of the SEQ ID No.: 1; according to the transcription +1 position of SEQ ID No.: 1 as a standard, SEQ ID No.: 2 is identified as from −1952 position to −1901 position in the SEQ ID No.: 1.), rs12313273 (SEQ ID No.: 3 is at position 30855 to position 30906 of the SEQ ID No.: 1; according to the transcription +1 position of SEQ ID No.: 1 as a standard, SEQ ID No.: 3 is identified as from −1664 position to −1613 position in the SEQ ID No.: 1.), rs7135617 (SEQ ID No.: 4 is at position 36850 to position 36901 of the SEQ ID No.: 1; according to the transcription +1 position of SEQ ID No.:1 as a standard, SEQ ID No.: 4 is identified as from 4332 position to 4383 position in the SEQ ID No.: 1.), rs6486795 (SEQ ID No.: 5 is at position 43762 to position 43813 of the SEQ ID No.: 1; according to the transcription +1 position of SEQ ID No.: 1 as a standard, SEQ ID No.: 5 is identified as from 11244 position to 11295 position in the SEQ ID No.: 1.) and rs712853 (SEQ ID No.: 6 is at position 47514 to position 47565 of the SEQ ID No.: 1; according to the transcription +1 position of SEQ ID No.: 1 as a standard, SEQ ID No.: 6 is identified as from 14996 position to 15047 position in the SEQ ID No.: 1.). The relative positions of the five single-nucleotide polymorphisms mentioned above are shown as FIG. 1.

rs12320939 (G/T) is at position 30593 of the SEQ ID No.: 1, rs12313273 (C/T) is at position 30881 of the SEQ ID No.: 1, rs7135617 (G/T) is at position 36876 of the SEQ ID No.: 1, rs6486795 (C/T) is at position 43788 of the SEQ ID No.: 1 and rs712853 (C/T) is at position 47593 of the SEQ ID No.: 1.

In one embodiment, determinations for genotypes of the five single-nucleotide polymorphisms mentioned above are performed to biosamples of a healthy group and atopic dermatitis group, respectively, and the results of the determinations are analyzed by statistics to confirm that rs12313273 (C/T) is a potent biomarker for determining the development of atopic dermatitis and the severity of atopic dermatitis.

Therefore, in one aspect of the invention, the invention relates to a method for determining a risk, for a subject, of suffering from atopic dermatitis comprising the steps as described in the following. First, a biosample of a subject is obtained. The subject may comprise a mammal. In one embodiment, the subject may comprise a human. The biosample of the invention may be collected or isolated from any source containing chromosomal DNA. The source may comprise blood or saliva. In one embodiment, the biosample may comprise blood or saliva, for example, about 3-5 c.c. of blood or saliva.

Then, the presence of the single-nucleotide polymorphism rs12313273 (C/T) at position 30881 of the ORAI1 gene (SEQ ID No.:1) in the biosample is detected. A method for detecting the single-nucleotide polymorphism rs12313273 (C/T) may comprise primer extension (such as PinPoint assay, Massextend™, SPC-SBE, or GOOD assay), hybridization (such as TaqMan assay, bead array, or single-nucleotide polymorphism chip), ligation (combinatorial fluorescence energy transfer (CFET) tags), and enzymatric cleavage (RFLP, Invader® assay), PCR-SSCP (single-strand conformation polymorphism), MRD (mismatch repair dection), BeadArray™, or SNPlex™. In one embodiment, a genotype of the single-nucleotide polymorphism rs12313273 may be identified with TaqMan assay. In one embodiment, the single-nucleotide polymorphism rs12313273 (C/T) is detected by using a first oligonucleotide and a second oligonucleotide, and the first oligonucleotide is used for detecting the T allele of the single-nucleotide polymorphism rs12313273 (C/T) and the second oligonucleotide is used for detecting the C allele of the single-nucleotide polymorphism rs12313273 (C/T). Finally, the risk, for the subject, of suffering from atopic dermatitis is determined and when the presence of a C allele or genotype CC of the single-nucleotide polymorphism rs12313273 (C/T) is detected, it indicates that the subject is at increased risk for suffering from atopic dermatitis.

In one embodiment, for suffering from atopic dermatitis, the increased risk mentioned above is a relative risk (odds ratio) of at least about 1.4, preferably at least about 1.44 when the C allele is compared with the T allele of the single-nucleotide polymorphism rs12313273 (C/T). In another embodiment, for suffering from atopic dermatitis, the increased risk mentioned above is a relative risk (odds ratio) of at least about 2.4, preferably at least about 2.48 when the genotype CC is compared with the genotype TT of the single-nucleotide polymorphism rs12313273 (C/T).

As mentioned above, it is clearly understood that the single-nucleotide polymorphism rs12313273 (C/T) may be used as one of the biomarkers for a biomarker for determining the development of atopic dermatitis.

Therefore, in another aspect of the invention, the invention relates to a method for using a single-nucleotide polymorphism rs12313273 (SEQ ID No.: 3) as a biomarker for determining the development of atopic dermatitis or a method for using a single-nucleotide polymorphism rs12313273 (SEQ ID No.: 3) as a biomarker for determining a risk, for a subject, of suffering from atopic dermatitis.

In addition, in further another aspect of the invention, the invention relates to a method for determining severity of atopic dermatitis for a subject suffering from atopic dermatitis comprising the steps as described in the following. First, a biosample of a subject suffering from atopic dermatitis is obtained. The subject suffering from atopic dermatitis may comprise a mammal. In one embodiment, the subject may comprise a human. The biosample of the invention may be collected or isolated from any source containing chromosomal DNA. The source may comprise blood or saliva. In one embodiment, the biosample may comprise blood or saliva, for example, about 3-5 c.c. of blood or saliva.

Then, the presence of the single-nucleotide polymorphism rs12313273 (C/T) at position 30881 of the ORAI1 gene (SEQ ID No.: 1) in the biosample is detected. A method for detecting the single-nucleotide polymorphism rs12313273 (C/T) may comprise a primer extenstion (such as PinPoint assay, Massextend™, SPC-SBE, or GOOD assay), hybridization (such as TaqMan assay, bead array, or single-nucleotide polymorphism chip), ligation (combinatorial fluorescence energy transfer (CFET) tags), and enzymatric cleavage (RFLP, Invader® assay), PCR-SSCP (single-strand conformation polymorphism), MRD (mismatch repair dection), BeadArray™, or SNPlex™. In one embodiment, a genotype of the single-nucleotide polymorphism rs12313273 may be identified with TaqMan assay. In one embodiment, the single-nucleotide polymorphism rs12313273 (C/T) is detected by using a first oligonucleotide and a second oligonucleotide, and the first oligonucleotide is used for detecting the T allele of the single-nucleotide polymorphism rs12313273 (C/T) and the second oligonucleotide is used for detecting the C allele of the single-nucleotide polymorphism rs12313273 (C/T).

Finally, the severity, for the subject, of suffering from atopic dermatitis is determined. According to the result of the detection, when the presence of the C allele of the single-nucleotide polymorphism rs12313273 (C/T) is detected, it indicates that the subject is at risk for increased severity of atopic dermatitis.

In one embodiment, when compared to detection of a genotype TT of the single-nucleotide polymorphism rs12313273 (C/T), when the genotype CT of the single-nucleotide polymorphism rs12313273 (C/T) is detected, the subject is at risk for increased severity of atopic dermatitis. In another embodiment, when compared to detection of a genotype CT of the single-nucleotide polymorphism rs12313273 (C/T), when the genotype CC of the single-nucleotide polymorphism rs12313273 (C/T) is detected, the subject is at risk for increased severity of atopic dermatitis. In further another embodiment, when compared to detection of a genotype TT of the single-nucleotide polymorphism rs12313273 (C/T), when the genotype CC of the single-nucleotide polymorphism rs12313273 (C/T) is detected, the subject is at risk for increased severity of atopic dermatitis.

In other words, when compared to detection of a patient with a genotype TT or CT of the single-nucleotide polymorphism rs12313273 (C/T), a patient with a genotype CC of the single-nucleotide polymorphism rs12313273 (C/T) has most severe atopic dermatitis. In one example, the scoring for severity of atopic dermatitis (SCORAD) for a subject with a genotype TT of the single-nucleotide polymorphism rs12313273 (C/T) who suffers from atopic dermatitis is about 23, the scoring for severity of atopic dermatitis (SCORAD) for a subject with a genotype CT of the single-nucleotide polymorphism rs12313273 (C/T) who suffers from atopic dermatitis is about 27, and the scoring for severity of atopic dermatitis (SCORAD) for a subject with a genotype CC of the single-nucleotide polymorphism rs12313273 (C/T) who suffers from atopic dermatitis is about 29.

As mentioned above, it is clearly understood that the single-nucleotide polymorphism rs12313273 (C/T) may be used as one of the biomarkers for determining the severity of atopic dermatitis.

Therefore, in further another aspect of the invention, the invention comprises a method for using a single-nucleotide polymorphism rs12313273 (SEQ ID No.: 3) as a biomarker for determining severity of atopic dermatitis or a method for using a single-nucleotide polymorphism rs12313273 (SEQ ID No.: 3) as a biomarker for determining severity of atopic dermatitis for a subject suffering from atopic dermatitis.

### EXAMPLES

siOrai1 Prevented EGF-Mediated COX-2 Gene Expression

1. Effect of siOrai1 Transfection on the Expression Level of an Orai1 Protein

Method:

A431 cells were transfected by ORAI1 siRNA (while the control was transfected by Control siRNA) and cultured for 48 hours, and then stimulated with EGF with a concentration of 25 ng/ml. Next, the total protein in the cells was obtained by using a RIPA buffer, and the expression amount of Orai1 protein in the cells was determined by Western blotting.

Result:

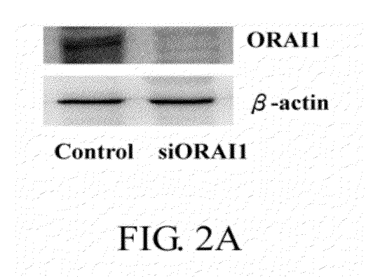

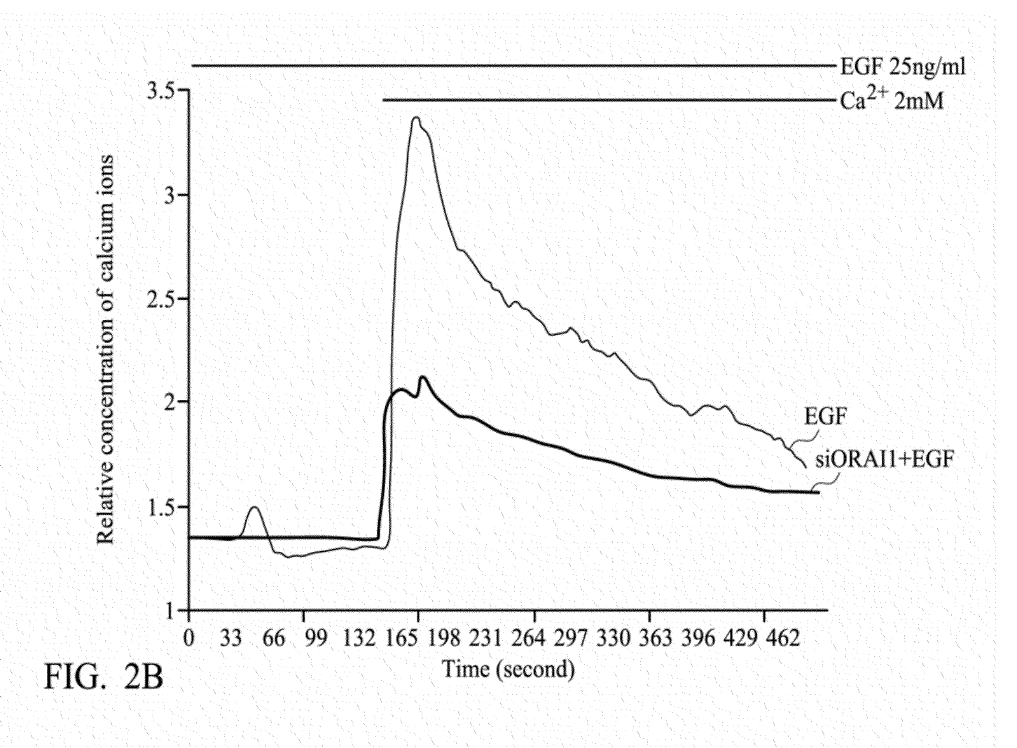

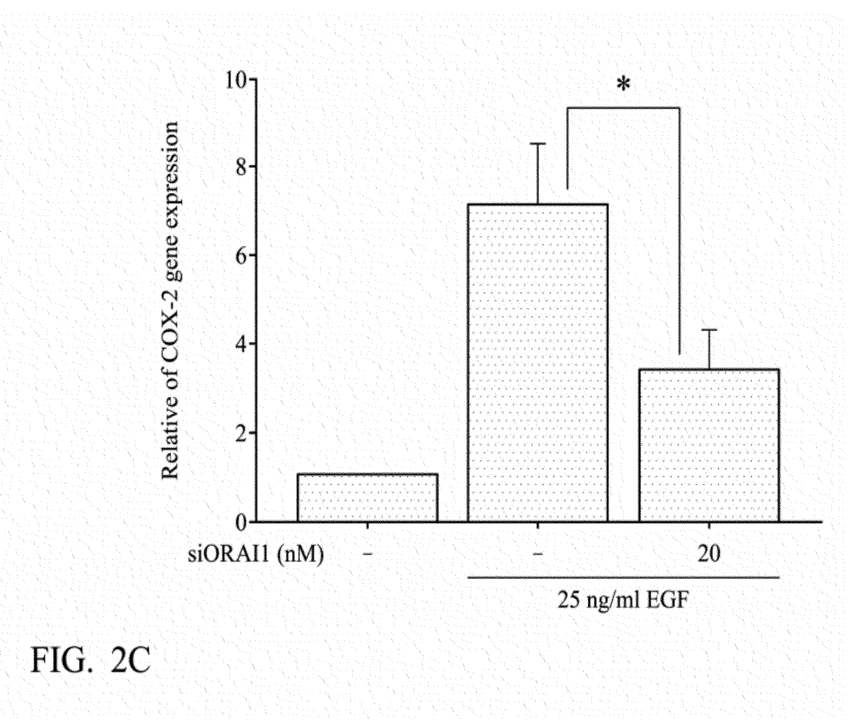

The result is shown as FIG. 2A. When the cells were treated with siOrai1, the gene expression of ORAI1 was inhibited.

2. siOrai1 Prevented EGF-Mediated Calcium Ion Channel Efficiency

Method

Signals for calcium ions passing through a store-operated Ca²⁺ channel were analyzed by real time imaging for calcium ions in the single cell. At room temperature (20-25° C.), an Olympus Cell R fluorescence microscopy system was used to record the results. All processes of the experiment were performed in the dark, and the cells were first treated with 1 μM Fluo-4 and then with a calcium ion solution to be detected for 7-8 minutes (Fluo-4 was used as a fluorescence agent, and the fluorescence intensity thereof represented the concentration of the calcium ions). The composition of the calcium ion solution (pH 7.4) comprised 145 mM sodium chloride, 5.4 mM potassium chloride, 2.0 mM calcium chloride, 2 mM magnesium sulfate, 20 mM HEPES, 5.5 mM D-glucose and sodium hydroxide.

Result:

The result is shown as FIG. 2B. As compared to the control group only treated with EGF, the calcium ion channel efficiency of the group treated with EGF and siOrai1 decreased, significantly.

3. siOrai1 Prevented EGF-Mediated COX-2 Inflammation Gene Expression

Method

A431 cells were transfected by ORAI1 siRNA (while the control was transfected by Control siRNA) and cultured for 48 hours, and then stimulated with EGF with a concentration of 25 ng/ml. Next, the total RNA of the cells was extracted by using a Trizol agent. After purifying mRNA from the total RNA, the mRNA was reverse-transcripted as cDNA to detect an amount of COX-2 gene expression by a SYBR Green assay.

Result:

The result is shown as FIG. 2C. As compared to the control group only treated with EGF, the COX-2 gene expression of the group treated with EGF and siOrai 1 was inhibited, significantly.

Relationship Between the Single-Nucleotide Polymorphism (SNP) and Atopic Dermatitis

1. Material and Method

(a) DNA Extraction for Biosample of a Subject

According the standard procedure provided by the Puregene™ kit from Gentra (Research Triangle, NC), 5 ml of venous whole blood was placed in a tube containing EDTA and centrifuged at 3000 rpm/min for 10 minutes to separate plasma, leukocyte buffy coat and erythrocytes. The buffy coat was taken out from the tube and added into a 1.5 ml microcentrifuge tube by a micropipette and three times that of the buffy coat volume of a RBC lysis solution was added into the microcentrifuge tube. The microcentrifuge tube was shaken to mix the buffer and the buffy coat well, and centrifuged at 13,000-16,000 rpm/min for 1 minute to precipitate the leukocyte containing DNA. Then, a supernatant therefrom was removed from the microcentrifuge tube and 600 μl of a cell lysis buffer was added thereto, and then the microcentrifuge tube was shaken to mix the remaining and the cell lysis buffer well and kept for least one night. After the cell membrane was broken and DNA was released from the cells, 300 μl of a protein precipitation solution was added into the microcentrifuge tube and mixed with the remaining. Next, the microcentrifuge tube was centrifuged at 13,000-16,000 rpm/min for 5 minutes to precipitate the impurity. A supernatant containing DNA therefrom was taken and added into a new microcentrifuge tube and an equal amount of isopropanol was added thereto and the new microcentrifuge tube was shaken, slightly. After that, the new microcentrifuge tube was centrifuged at 13,000-16,000 rpm/min for 2 minutes to obtain a white precipitate containing DNA and a supernatant. The supernatant was removed carefully and 450 μl of ethanol was added into the microcentrifuge tube. Then, the new microcentrifuge tube was centrifuged at 13,000-16,000 rpm/min for 3 minutes and a supernatant therefrom was removed to remove the undesired salts. The ethanol remained in the new microcentrifuge tube was dried to completely evaporate and then 100 μl of d. d. water was added into the new microcentrifuge tube. Then, the new microcentrifuge tube was placed in a 65° C. water bath for 5 minutes to accelerate the DNA being redissolved in the d. d. water.

The concentration and purity (O.D. 260/280) of the obtained DNA was determined by a spectrophotometer and then diluted to a concentration of 10 ng/μl for use in the determination of a genotype.

(b) Selection Conditions for Single-Nucleotide Polymorphisms

The single-nucleotide polymorphisms of the invention were selected from the data base Phase III of the International Haplotype HapMap project (http://www.hapmap.org). The Haplotype HapMap project was co-established by the governments of the United State of America, China, Canada, Japan, Nigeria and Britain, for the purpose of identifying and cataloging genetic information of human beings, such as those related to single-nucleotide polymorphisms, from different ethnic groups, populations, and geographic locations. The Haplotype HapMap project encompasses Nigerian-Americans, Japanese-Americans, Chinese-Americans and European-Americans, wherein the frequencies for single-nucleotide polymorphisms distributed among different populations can be accessed.

The selection standard for single-nucleotide polymorphisms in the invention is that the single-nucleotide polymorphism must meet r²≧0.8 and the minor allele frequency (MAF) for Han Chinese in Beijing 10%, wherein the selected single-nucleotide polymorphisms are all representative tagging single-nucleotide polymorphisms (tSNPs) in linkage disequilibrium in the genomic regions. According to the standard, five tagging single-nucleotide polymorphisms were selected from the ORAI1 gene (SEQ ID. No.: 1) in the invention. The five tagging single-nucleotide polymorphisms were rs 12320939 (SEQ ID No.: 2 is at position 30567 to position 30618 of the SEQ ID No.: 1), rs12313273 (SEQ ID No.: 3 is at position 30855 to position 30906 of the SEQ ID No.: 1), rs7135617 (SEQ ID No.: 4 is at position 36850 to position 36901 of the SEQ ID No.: 1), rs6486795 (SEQ ID No.: 5 is at position 43762 to position 43813 of the SEQ ID No.: 1) and rs712853 (SEQ ID No.: 6 is at position 47514 to position 47565 of the SEQ ID No.: 1), respectively.

(c) Genotype Determination

TaqMan technique (TaqMan Genotyping Assays products, Applied Biosystems Inc.) was used to perform the genotype determination in the invention. In the TaqMan technique, two probes (Minor Grove Binder probe) carrying different fluorescent dyes (FAM™ dye and VIC™ dye) at their 5′ ends, respectively are used. Due to the difference of the designed sequences for the two probes, the two probes are able to match with the two different alleles of a single-nucleotide polymorphism, respectively. In addition, a non-fluorescent quencher (NFQ) at the 3′ end of the respective probe is used to absorb the fluorescence energy of the fluorescent dye so that no fluorescent light could be release from the fluorescent dye. Next, the polymerase (AmpliTaq Gold DNA polymerase) separate the fluorescent dye from the respective probe which is attached to the complementary sequence and from the non-fluorescent quencher via a polymerase chain reaction, and the fluorescent dye at the 5′ end of the respective probe can begin to release fluorescence. According to the different fluorescence being detected, different genotypes were able to be identified. In the experiment, the TaqMan® Genotyping Assays rs12320939 C_{—}31833529_{—}10 was used to detect rs12320939, the TaqMan® Genotyping Assays rs12313273 C_{—}31833531_{—}10 was used to detect rs12313273, the TaqMan® Genotyping Assays rs7135617 C_{—}248428_{—}10 was used to detect rs7135617, the TaqMan® Genotyping Assays rs6486795 C_{—}1596513_{—}20 was used to detect rs6486795, and the Custom Taqman® SNP Genotyping Assay Service rs712853 was used to detect rs712853.

The real-time polymerase chain reaction machine (ABI 7500, Applied Biosystems Inc, Foster City, Calif.) was used to perform the polymerase chain reaction and genotype determination in the invention. 2.5 μl of TaqMan universal Master Mix, 0.0625 μl of 40× TaqMan Genotyping Assay primer-probe Mix, 1 μl of DNA and 1.4375 μl of d. d. d. water constituted each sample with a total volume of 5 μl. Each mixed sample was placed in a 96 well reaction plate. The polymerase chain reaction for each sample was performed in the real-time polymerase chain reaction machine with the reaction conditions as follow: 95° C. for 10 minutes; 95° C. for 15 seconds and 60° C. for 1 minutes for 45 cycles. The software designed by ABI was used to determine the genotype according to the fluorescence intensity detected.

(d) Statistics Analysis

SAS software vision 9.1 was used to perform the statistics analysis in the invention. Chi-square Goodness-of-fit test of Chi-square test was used to determine whether each tagging single-nucleotide polymorphism met the Hardy-Weinberg equilibrium (HWE) and when the p value was greater than 0.05, it indicated that there was no deviation in the genotype distribution. The chi-square test was used to determine whether there was significant difference between the distribution for each genotype and allele of patients with atopic dermatitis and those of the healthy control group. Student's t-test was used to determine IgE whether the difference between patients with atopic dermatitis having different genotypes was significant.

2. Relationship Between the Single-Nucleotide Polymorphism of ORAI1 Gene And Atopic Dermatitis

(a) Atopic Dermatitis Patient Group and Healthy Group

A case-control study of 102 patients with atopic dermatitis and 715 healthy people were selected for the study of the invention, respectively. The detailed information for the atopic dermatitis patient group and healthy group are shown as Table 1.

TABLE 1
Detailed information for the atopic dermatitis patient group and
healthy group

| | Case (n) (%) | Control (n) (%) |
|---|---|---|
| Numbers | 102 | 715 |
| Sex: male | 66 (64.7%) | 401 (56.1%)^{a} |
| Age: year^{b} | 53.0 ± 23.5 | 39.9 ± 19.1^{c} |
| Age range: year | 4-91 | 11-85 |
| Adult atopic dermatitis | 87 (85.3%) | |
| Atopic dermatitis | 15 (14.7%) | |

^{a}P = 0.11;
^{b}Mean ± Standard deviation;
^{c}P < 0.0001

(b) Analysis for Relationship Between the rs12320939 and the Development of Atopic Dermatitis

The rs12320939 genotypes of 101 patients with atopic dermatitis and 678 healthy people were studied. The results are shown in Table 2.

TABLE 2
Relationship between the rs12320939 and the development of atopic
dermatitis

| | | | | Odds ratio (OR) |
|---|---|---|---|---|
| | | Case (n) (%) | Control (n) (%) | (95% C.I.) |
| Numbers | | 101 | 678 | |
| ORAI1- | TT | 32 (31.7%) | 165 (24.3%) | 1.29 (0.74-2.24) |
| rs12320939 | GT | 42 (41.6%) | 334 (49.3%) | 0.83 (0.50-1.40) |
| | GG | 27 (26.7%) | 179 (26.4%) | 1.00 (Reference) |
| | T | 106 (52.5%) | 664 (49.0%) | 1.15 (0.86-1.55) |
| | G | 96 (47.5%) | 692 (51.0%) | 1.00 (Reference) |

Overall P value=0.2284

Trend P=0.3604

Dominant model P value=0.9438; Odds ratio (OR)=0.98

Recessive model P value=0.1130; Odds ratio (OR)=1.44

(c) Analysis for Relationship Between the rs12313273 and the Development of Atopic Dermatitis

The rs12313273 genotypes of 102 patients with atopic dermatitis and 687 healthy people were studied. The results are shown in Table 3.

TABLE 3
Relationship between the rs12313273 and the development of atopic
dermatitis

| | | | | Odds ratio (OR) |
|---|---|---|---|---|
| | | Case (n) (%) | Control (n) (%) | (95% C.I.) |
| Numbers | | 102 | 687 | |
| ORAI1- | CC | 17 (16.7%) | 53 (7.7%) | 2.48 (1.33-4.63) |
| rs12313273 | CT | 38 (37.2%) | 271 (39.5%) | 1.08 (0.69-1.71) |
| | TT | 47 (46.1%) | 363 (52.8%) | 1.00 (Reference) |
| | C | 72 (35.3%) | 377 (27.4%) | 1.44 (1.06-1.97) |
| | T | 132 (64.7%) | 997 (72.6%) | 1.00 (Reference) |

Overall P value=0.0116

Trend P=0.0228

Dominant model P value=0.2023; Odds ratio (OR)=1.31

Recessive model P value=0.0030; Odds ratio (OR)=2.39

(d) Analysis for Relationship Between the rs7135617 and the Development of Atopic Dermatitis

The rs7135617 genotypes of 101 patients with atopic dermatitis and 687 healthy people were studied. The results are shown in Table 4.

TABLE 4
Relationship between the rs7135617 and the development of atopic
dermatitis

| | | | | Odds ratio (OR) |
|---|---|---|---|---|
| | | Case (n) (%) | Control (n) (%) | (95% C.I.) |
| Numbers | | 101 | 687 | |
| ORAI1- | TT | 21 (20.8%) | 113 (16.4%) | 0.96 (0.55-1.68) |
| rs7135617 | GT | 34 (33.7%) | 337 (49.1%) | 0.52 (0.32-0.83) |
| | GG | 46 (45.5%) | 237 (34.5%) | 1.00 (Reference) |
| | T | 76 (37.6%) | 563 (41.0%) | 0.87 (0.64-1.18) |
| | G | 126 (62.4%) | 811 (59.0%) | 1.00 (Reference) |

Overall P value=0.0150

Trend P=0.3706

Dominant model P value=0.0307; Odds ratio (OR)=0.63

Recessive model P value=0.2779; Odds ratio (OR)=1.33

(e) Analysis for Relationship Between the rs6486795 and the Development of Atopic Dermatitis

The rs6486795 genotypes of 93 patients with atopic dermatitis and 683 healthy people were studied. The results are shown in Table 5.

TABLE 5
Relationship between the rs6486795 and the development of atopic
dermatitis

| | | | | Odds ratio (OR) |
|---|---|---|---|---|
| | | Case (n) (%) | Control (n) (%) | (95% C.I.) |
| Numbers | | 93 | 683 | |
| ORAI1- | CC | 23 (24.7%) | 98 (14.4%) | 2.04 (1.14-3.68) |
| rs6486795 | CT | 39 (42.0%) | 315 (46.1%) | 1.08 (0.65-1.78) |
| | TT | 31 (33.3%) | 270 (39.5%) | 1.00 (Reference) |
| | C | 85 (45.7%) | 511 (37.4%) | 1.41 (1.03-1.92) |
| | T | 101 (54.3%) | 855 (62.6%) | 1.00 (Reference) |

Overall P value=0.0336

Trend P=0.0321

Dominant model P value=0.2498; Odds ratio (OR)=1.31

Recessive model P value=0.0096; Odds ratio (OR)=1.96

(f) Analysis for Relationship Between the rs712853 and the Development of Atopic Dermatitis

The rs712853 genotypes of 101 patients with atopic dermatitis and 689 healthy people were studied. The results are shown in Table 6.

TABLE 6
Relationship between the rs712853 and the development of atopic
dermatitis

| | | | | Odds ratio (OR) |
|---|---|---|---|---|
| | | Case (n) (%) | Control (n) (%) | (95% C.I.) |
| Numbers | | 101 | 689 | |
| ORAI1- | CC | 14 (13.9%) | 96 (13.9%) | 0.81 (0.43-1.53) |
| rs712853 | CC | 14 (13.9%) | 96 (13.9%) | 0.81 (0.43-1.53) |
| | CT | 34 (33.6%) | 297 (43.1%) | 0.64 (0.40-1.01) |
| | TT | 53 (52.5%) | 296 (43.0%) | 1.00 (Reference) |
| | C | 62 (30.7%) | 489 (35.5%) | 0.81 (0.59-1.11) |
| | T | 140 (69.3%) | 889 (64.5%) | 1.00 (Reference) |

Overall P value=0.1558

Trend P=0.1984

Dominant model P value=0.0721; Odds ratio (OR)=0.68

Recessive model P value=0.9845; Odds ratio (OR)=0.99

According to the results mentioned above, it was discovered that the polymorphism positions, rs12313273, rs6486795 and rs7135617, were related to the development of atopic dermatitis. When the rs12313273 CC genotype carriers were compared to the rs12313273 TT genotype carriers, the odds ratio was 2.48 (95% C.I: 1.33-4.63). Also, the risk for the development of atopic dermatitis for the rs6486795 CC genotype carriers was 2.04-fold that of the rs6486795 TT genotype carriers (95% C.I: 1.14-3.68). Meanwhile, the rs7135617 TT or GT genotype has protective effect preventing from development of atopic dermatitis.

3. Relationships Between the Severity of Atopic Dermatitis and the Single-Nucleotide Polymorphisms of ORAI1 Gene

An analysis for the genotype of rs12313273 was performed to 102 patients with atopic dermatitis, an analysis for genotype of rs6486795 was performed to 93 patients with atopic dermatitis and an analysis for genotype of rs7135617 was performed to 101 patients with atopic dermatitis, and then severity of atopic dermatitis, for patients having different genotypes, was determined. The results are shown in Table 7.

TABLE 7
Difference in severity of atopic dermatitis, for patients having different
genotypes

| | | | | P value |
|---|---|---|---|---|
| | ORAI1-rs12313273 | | | |
| Genotype | CC | CT | TT | |
| Numbers | 17 | 38 | 47 | |
| SCORAD | 29.5 ± 10.3 | 27.7 ± 8.4 | 23.7 ± 9.6 | 0.04 |
| | ORAI1-rs6486795 | | | |
| Genotype | CC | CT | TT | |
| Numbers | 23 | 39 | 31 | |
| SCORAD | 29.3 ± 10.8 | 25.5 ± 6.9 | 23.2 ± 9.5 | 0.05 |
| | ORAI1-rs7135617 | | | |
| Genotype | TT | GT | GG | |
| Numbers | 21 | 34 | 46 | |
| SCORAD | 23.8 ± 10.0 | 25.4 ± 9.3 | 27.5 ± 9.3 | 0.29 |

Table 7 shows that as compared to the rs12313273 CT or TT genotype carriers, the rs12313273 CC genotype carriers had higher severity of the disease (29.5 vs. 27.7 vs. 23.7; P=0.04).

Therefore, according to all of the analytical results mentioned above, it is clearly shown that the single-nucleotide polymorphism rs12313273 of the ORAI1 gene is able to be used as a biomarker for determining the severity of atopic dermatitis.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for determining a risk, for a subject, of suffering from atopic dermatitis, comprising:
obtaining a biosample of the subject;
detecting the presence of the single-nucleotide polymorphism rs12313273 (C/T) at position 30881 of the ORAI1 gene (SEQ ID No.: 1) in the biosample; and
determining the risk, for the subject, of suffering from atopic dermatitis, wherein the presence of a C allele or genotype CC of the single-nucleotide polymorphism rs12313273 (C/T) indicates that the subject is at increased risk for suffering from atopic dermatitis.

2. The method for determining a risk, for a subject, of suffering from atopic dermatitis as claimed in claim 1, wherein the increased risk is a relative risk of at least about 1.4 when the C allele is compared with the T allele of the single-nucleotide polymorphism rs12313273 (C/T).

3. The method for determining a risk, for a subject, of suffering from atopic dermatitis as claimed in claim 1, wherein the increased risk is a relative risk of at least about 2.4 when the genotype CC is compared with the genotype TT of the single-nucleotide polymorphism rs12313273 (C/T).

4. The method for determining a risk, for a subject, of suffering from atopic dermatitis as claimed in claim 1, wherein the subject comprises a mammal.

5. The method for determining a risk, for a subject, of suffering from atopic dermatitis as claimed in claim 1, wherein the subject comprises a human.

6. The method for determining a risk, for a subject, of suffering from atopic dermatitis as claimed in claim 1, wherein the biosample comprises blood or saliva.

7. The method for determining a risk, for a subject, of suffering from atopic dermatitis as claimed in claim 1, wherein the single-nucleotide polymorphism rs12313273 (C/T) is detected by using a first oligonucleotide and a second oligonucleotide, and the first oligonucleotide is used for detecting the T allele of the single-nucleotide polymorphism rs12313273 (C/T) and the second oligonucleotide is used for detecting the C allele of the single-nucleotide polymorphism rs12313273 (C/T).

8. A method for determining severity of atopic dermatitis for a subject suffering from atopic dermatitis, comprising:
obtaining a biosample of the subject suffering from atopic dermatitis;
detecting the presence of the single-nucleotide polymorphism rs12313273 (C/T) at position 30881 of the ORAI1 gene (SEQ ID No.: 1) in the biosample; and
determining the severity, for the subject, of suffering from atopic dermatitis, wherein the presence of the C allele of the single-nucleotide polymorphism rs12313273 (C/T) indicates that the subject is at risk for increased severity of atopic dermatitis.

9. The method for determining severity of atopic dermatitis for a subject suffering from atopic dermatitis as claimed in claim 8, wherein when compared to detection of a genotype TT of the single-nucleotide polymorphism rs12313273 (C/T), when the genotype CT of the single-nucleotide polymorphism rs12313273 (C/T) is detected, the subject is at risk for increased severity of atopic dermatitis.

10. The method for determining severity of atopic dermatitis for a subject suffering from atopic dermatitis as claimed in claim 8, wherein when compared to detection of a genotype CT of the single-nucleotide polymorphism rs12313273 (C/T), when the genotype CC of the single-nucleotide polymorphism rs12313273 (C/T) is detected, the subject is at risk for increased severity of atopic dermatitis.

11. The method for determining severity of atopic dermatitis for a subject suffering from atopic dermatitis as claimed in claim 8, wherein when compared to detection of a genotype TT of the single-nucleotide polymorphism rs12313273 (C/T), when the genotype CC of the single-nucleotide polymorphism rs12313273 (C/T) is detected, the subject is at risk for increased severity of atopic dermatitis.

12. The method for determining severity of atopic dermatitis for a subject suffering from atopic dermatitis as claimed in claim 8, wherein the subject suffering from atopic dermatitis comprises a mammal.

13. The method for determining severity of atopic dermatitis for a subject suffering from atopic dermatitis as claimed in claim 8, wherein the subject suffering from atopic dermatitis comprises a human.

14. The method for determining severity of atopic dermatitis for a subject suffering from atopic dermatitis as claimed in claim 8, wherein the biosample comprises blood or saliva.

15. The method for determining severity of atopic dermatitis for a subject suffering from atopic dermatitis as claimed in claim 8, wherein the single-nucleotide polymorphism rs12313273 (C/T) is detected by using a first oligonucleotide and a second oligonucleotide, and the first oligonucleotide is used for detecting the T allele of the single-nucleotide polymorphism rs12313273 (C/T) and the second oligonucleotide is used for detecting the C allele of the single-nucleotide polymorphism rs12313273 (C/T).

16. A method for using a single-nucleotide polymorphism rs12313273 (SEQ ID No.: 3) as a biomarker for determining the development of atopic dermatitis.

17. A method for using a single-nucleotide polymorphism rs12313273 (SEQ ID No.: 3) as a biomarker for determining the severity of atopic dermatitis.
